(19) United States
(12) Patent Application Publication
(10) Pub. No.: US 2024/0374781 A1
(43) Pub. Date: Nov. 14, 2024

(54) PATCH COMPRISING SENSING COMPONENTS

(71) Applicants: EMPA EIDGENÖSSISCHE MATERIALPRÜFUNGS-UND FORSCHUNGSANSTALT, Dübendorf (CH); ETH ZURICH, Zürich (CH)

(72) Inventors: Alexandre ANTHIS, Zürich (CH); Inge HERRMANN, Siebnen/Wangen (CH); Benjamin SUTER, Muntelier (CH)

(73) Assignees: EMPA EIDGENÖSSISCHE MATERIALPRÜFUNGS-UND FORSCHUNGSANSTALT, Dübendorf (CH); ETH ZURICH, Zürich (CH)

(21) Appl. No.: 18/272,705

(22) PCT Filed: Jan. 19, 2022
(86) PCT No.: PCT/EP2022/051141

§ 371 Date: Jul. 17, 2023

(30) Foreign Application Priority Data
Jan. 19, 2021 (EP) 21152240.4

(51) Int. Cl. A61L 24/00

(52) U.S. Cl. CPC A61L 24/0031; A61L 2300/102; A61L 2300/232

(57) ABSTRACT

A patch for detecting by at least one non-invasive imaging technique a leak of a biological fluid at an application site, wherein the patch includes either a layer including one or more first portions containing a first gel, one or more second portions containing a second gel and at least one sensing component embedded within the first gel and/or the second gel, or a layer containing a hydrogel and at least one sensing component, such as aerogel particles, and xerogel particles, embedded within the hydrogel.

### FIELD OF THE INVENTION

The present invention relates to the field of surgery, in particular to a patch for monitoring of an application site, such as a sutured site, a stapled site or an implantation site of a surgical implant. The invention provides a patch for detecting a leak of a biological fluid at an application site, such as a sutured site, a stapled site or an implantation site of a surgical implant.

### BACKGROUND OF THE INVENTION

Many diseases, including colorectal cancers, bowel ischemia and inflammatory diseases such as Crohn's disease or ulcerative colitis, often require invasive surgical procedures. Typically, such surgeries include removing or circumventing diseased tissue and connecting the remaining healthy tissue extremities by suturing or stapling, resulting in anastomosis sites. Such abdominal organ anastomoses can be accompanied by severe complications. Of these, anastomotic leakage is one of the most dreaded complications with reported incidence rates ranging from 4 to 21%, depending on the patient's condition (e.g. the patient's sarcopenia level) and the surgeon's experience. Reported mortality rates for patients suffering from anastomotic leaks range from 6 to 27%. In case of progression to septic peritonitis due to leaking of the bacteria-containing intestinal fluid, mortality rates as high as 50% have been reported.

While major efforts have focused on the prevention of such leaks, once established, their treatment is especially costly (usually more than 30,000 USD), and lengthy (usually more than 10 days in hospital). Furthermore, the treatment of anastomotic leaks often requires the formation of temporary stomas and artificial drains to the patient's body for monitoring, adding to the societal burden that complications particularly during the healing of abdominal fistulas impose on the patient.

There is an unmet medical need to provide diagnostic materials that enable monitoring of a sutured site, a stapled site, or an implantation site of a surgical implant by non-invasive imaging-techniques.

### DESCRIPTION OF THE INVENTION

Thus, it is an object of the present invention to address this need. The objective is achieved by a patch as defined in claim 1. Preferred embodiments are disclosed in the specification and the dependent claims.

The present invention will be described in more detail below. Where the present description refers to “preferred” embodiments/features, combinations of these “preferred” embodiments/features are also deemed to be disclosed as long as the specific combination of the “preferred” embodiments/features is technically meaningful.

Unless otherwise stated, the following definitions shall apply in this specification:

As used herein, the term “a”, “an”, “the” and similar terms used in the context of the present invention (especially in the context of the claims) are to be construed to cover both the singular and plural unless otherwise indicated herein or clearly contradicted by the context.

As used herein, the term “and/or” means that either all or only one of the elements of said group may be present. For example, “A and/or B” means “only A, or only B, or both A and B”. In the case of “only A”, the term also covers the possibility that B is absent, i.e. “only A, but not B”.

As used herein, the terms “including”, “containing” and “comprising” are used herein in their open-ended, non-limiting sense. It is understood that the various embodiments, preferences and ranges may be combined at will. Thus, for instance a solution comprising a compound A may include other compounds besides A. However, the term “comprising” also covers, as a particular embodiment thereof, the more restrictive meanings of “consisting essentially of” and “consisting of, so that for instance “a solution comprising A, B and optionally C” may also (essentially) consist of A and B, or (essentially) consist of A, B and C.

The terms “treating”, “treat” and “treatment” include one or more of the following: (i) preventing a disease, pathologic or medical condition from occurring (e.g. prophylaxis); (ii) inhibiting the disease, pathologic or medical condition or arresting its development; (iii) relieving the disease, pathologic or medical condition; (iv) diminishing symptoms associated with the disease, pathologic or medical condition; (v) monitoring the application site. Thus, the terms “treat”, “treatment”, and “treating” extend to prophylaxis and include prevent, prevention, preventing, lowering, stopping or reversing the progression or severity of the condition or symptoms being treated. As such, the term “treatment” includes medical, therapeutic, and/or prophylactic administration, as appropriate.

The term “surgery” is known in the field and relates to surgical procedures involving operative manual and/or instrumental techniques on a person to investigate or treat a pathological condition such as a disease or injury, to help improve bodily function or appearance or to repair unwanted ruptured areas. In specific embodiments, the term surgery relates to resection, i.e. partial removal of an organ or other bodily structure. Resection of organs such as intestines involves their reconnection. Typically, internal suturing or stapling is used for reconnection. Surgical connection between blood vessels or other tubular or hollow structures such as loops of intestine is called anastomosis. Thus, in specific embodiments, the term surgery relates to anastomosis. In some cases, surgical procedures involve creation of artificial stoma that can be temporary or permanent and sealing of such stoma. Thus, in specific embodiments, the term surgery relates to sealing of artificial stoma, particularly temporary or permanent stoma.

The term “non-degradable” as used in the context of the present invention relates to the stability of a material when in contact with biological fluids under physiological conditions. More specifically, within the scope of the present invention, a nondegradable hydrogel layer, aerogel layer, xerogel layer, sensing component refers to a hydrogel layer, aerogel layer, xerogel layer, sensing component of which at least 80% remains intact upon incubation for 24 h at 37° C. in simulated intestinal fluid (SIF) as prepared according to a protocol from the united states pharmacopoeia (Test Solutions, United States Pharmacopeia 30, NF 25, 2007), compared to incubation in PBS. During incubation, a polymer and/or a sensing component contained by the hydrogel layer, aerogel layer, or xerogel layer may be degraded.

As used herein, the terms “degradable” and “digestible” refer to a material (e.g.: gel, sensing component), wherein at the most 20%, preferably less than 10%, more preferably less than 5% remains intact upon incubation for 24 h at 37° C. in simulated intestinal fluid (SIF) as prepared according to a protocol from the united states pharmacopoeia (Test Solutions, United States Pharmacopeia 30, NF 25, 2007), compared to incubation in PBS. Within the present patent application, the terms “degradable” and “digestible”, are used interchangeably.

The term “synthetic” as used in the context of the present invention relates to material which is not of biological origin, e.g. synthetic polymers. A synthetic hydrogel may comprise components that are of biological origin, however the synthetic hydrogel as such is not of biological origin.

Throughout this specification a number
of abbreviations are used, including:

| | |
|---|---|
| AA | Acrylic acid |
| AAm | acrylamide |
| BAC | N,N′-bis(acryloyl)cystamine |
| CBX-SS | carboxybetaine disulphide crosslinker |
| CMC | carboxymethylcellulose |
| CT | computer tomography |
| DMAEP | 2,2-dimethacroyloxy-1-ethoxypropane |
| HSA | human serum albumin |
| MA | methyl acrylate |
| mBAA | N,N′-Methylenebisacrylamide |
| mIPN | mutually interpenetrating network |
| MPC | methacryloyloxyethyl phosphorylcholine |
| MRI | magnetic resonance imaging |
| NHS | n-hydroxysuccinimide |
| NVA | N-vinyl acetamide |
| OD | optical density |
| PAMPS | poly(2-acrylamido-2-methyl-1-propanesulfonic acid) |
| PBS | phosphate buffered saline |
| PCL | polycaprolactone |
| PEG | polyethylenglycol |
| PEGDMA | polyethylene glycol dimethacrylate |
| PHEMA | poly(2-hydroxyethyl methacrylate) |
| PHPMA | poly(N-(2-hydroxypropyl)methacrylamide) |
| PNAGA | poly N-acryloyl glycinamide |
| PNHEA | poly(N-hydroxyethyl acrylamide |
| PHNEA-BAC | poly(N-hydroxyethyl acrylamide) crosslinked with |
| | N,N′-bis(acryloyl)cystamine |
| PVA | polyvinylacetate |
| PVDF | polyvinylidene difluoride |
| RFID | radio-frequency identification |
| SIF | simulated intestinal fluid |
| TEMED | N,N,N′,N′-Tetramethylethan-1,2-diamin |
| TriMPTA | 1,1,1-trimethylolpropane triacrylate |
| VEGF | vascular endothelial growth factor |
| wt % | weight % |
| vol % | volume % |
| LAP | lithium phenyl-2,4,6-trimethylbenzoylphosphinate |
| PAAm | polyacrylamide |
| BSA | bovine serum albumin |
| I2959 | Irgacure D-2959 |

### DESCRIPTION OF THE FIGURES

FIG. 1: Cross-section of representative patches comprising a layer with first portions containing a first gel and second portions containing a second gel as described herein: (I) first gel, (II) second gel, (III) sensing component 1, (IV) sensing component 2.

FIG. 2: Cross-section of a representative patch comprising a third gel and aerogel particles or xerogel particles as sensing component: (V) third gel, (VI) aerogel particles or xerogel particles.

FIG. 3: Representative patches and hydrogel layer without sensing component for comparison.
(a) Hydrogel layer without sensing components for comparison. Hydrogel layer prepared according to example 1.
(b) Patch comprising (i) a first gel (poly(acrylamide-co-methyl acrylate-co-acrylic acid)), wherein a second gel (PAAm) is encapsulated. The second gel contains gascontaining vesicles (20% vol gas-containing vesicles; sensing component for generating turn-off type signal). Gas-containing vesicles embedded in a PAAm gel are described in example 3a.
(c) Two disk shaped patches comprising a sensing component (left). Right: (i) Second gel having (glutaraldehyde-crosslinked 15 wt % bovine serum albumin) having (ii) 20% vol gas-containing vesicles (sensing component for generating turn-off type signal) embedded therein for the second portions of the patch claimed herein.

FIG. 4: Top, bottom left: Rectangular layered patch consisting of (i) backing layer containing PNHEA 50 wt %, (ii) 20 wt % Halo gas vesicle (sensing component) embedded in a second gel (Polyacrylamide), (iii) first gel (50 wt % PAMPS) without sensing component. This is shown in example 6a.

Bottom right: Layered circular patch comprising (i)-(iii) as in the top and bottom left gels but additionally containing (iv) 2.5 wt % ZnO (ZnO being both sensing component and therapeutic component).

FIG. 5: (a) Normalized absorbance spectrum of a 40 vol % (squares), 20 vol % (circles) gas-containing vesicle suspension in presence of enzymatically active simulated intestinal fluid compared to enzyme deficient simulated intestinal fluid as control (triangles), as function of time in hours. (b) Ultrasound image of “turn-off” sensing component in a first gel obtained using a Clarius L15HD probe (i) before and (ii) after contact with enzyme active intestinal fluid. The sensing component is 20 vol % gascontaining vesicles embedded in 20 wt % in a first gel (polyacrylamide; black background) as described in example 3a (50 μL of mixture used for mold-curing). (c) UItrasound image of a “turn-on” sensing component obtained using a Clarius L15HD probe (i) before and (ii) after contact with simulated gastric fluid. The sensing component is NaHCO₃ embedded in a second gel containing agar, which is encapsulated in a first gel (polyacrylamide) as described in example 4a. (d) Ultrasound image of a layered patch (rectangular shape) comprising a first gel (poly(N-acryloyl glycinamide)), a second gel (polyacrylamide) and a backing layer. A sensing component (“turn-off”-type sensing component; 20% vol gas-containing vesicles) is embedded in the second gel. The patch was attached to porcine small intestine. This example shows that gas-containing vesicles are detectable under ultrasound imaging. The backing layer contains polyacrylamide. The image was obtained using a Clarius L15HD probe.

FIG. 6: Sensing/Therapeutic component release and ultrasound imaging from a first gel. (a) Release percentage (y-axis) as a function of time (in hours) for ZnO nanoparticle sensing/therapeutic components from a first gel. 1 mg/mL ZnO nanoparticle were embedded in a first gel containing polyacrylamide (example 7) and incubated in (i) SIF, (ii) PBS and (iii) bacterial growth medium (tryptic soy broth). (b) Ultrasound image indicating the detectability of 10 wt % ZnO nanoparticle (2.5 mg/ml ZnO) embedded in a first gel (20 wt % AAm). This is shown in example 7. The first gel containing ZnO is embedded in an acrylamide imaging phantom. Ultrasound images were obtained using a Clarius L15HD probe.

FIG. 7: Ultrasound images performed using a Clarius 7HD probe controlled by an Ipad, performed at discrete time intervals (1, 3 and 6 hours) of a patterned circular sensing patch containing a first gel (PAMPS 50 wt %) and a second gel (polyacrylamide) wherein gas-containing vesicles are embedded in the second gel (period arrangement of 5 μL of 20 vol % gas-containing vesicles in PAAm matrix) and a backing layer containing PNHEA. This is shown in example 6a. The patch covers an intestine filled with simulated microbially active and enzyme rich digestive fluid. Condition (I) refers to the absence of a perforation on the tissue wall and (II) to the presence of a perforation at the site of the patch. Loss of contrast can be observed in (II) as a consequence of gastrointestinal fluid contact with the sensing elements, indicating the leak. In absence of a leak, contrast remains unchanged.

FIG. 8: Sensing component comprising lipid microbubbles (sensing component) (a) (Left) Second Harmonic backscatter image of SonoVue® (sensing component) in 20 wt. % Acrylamide Hydrogel (second gel) as described in example 8a. Incident ultrasound Freq. was 3.4 Hz, receiving Freq. was 6.8 Hz. Mechanical Index was set to 0.2

(Center) Second Harmonic backscatter image of pure 20 wt. % Acrylamide Hydrogel (no sensing component) at MI of 0.1 for comparison. Black region shows no signal being generated. Incident and receiving frequency identical to above.

(Right) Conventional ultrasound backscatter image (incident Freq=receiving Freq.) of SonoVue® (sensing component) in 20 wt. % Acrylamide Hydrogel (first gel).

(b) Long-term stability of SonoVue® (sensing component) in gel, images show signal after 12 days. SonoVue®-containing hydrogels were stored at room temperature. Gel composition as described in example 8b.

(Left) 20 μL SonoVue® (sensing component) diluted in 80 μL 20 wt % Acrylamide hydrogel (first gel).

(Center) 20 wt. % Acrylamide Hydrogel directly dissolved in 100 μL SonoVue® shows strong contrast

(Right) 100 μL SonoVue® (sensing component) diluted in 80 μL 20 wt. % Acrylamide Hydrogel (first gel).

A signal is well-discernable for all of the above conditions, whilst most pronounced in the center image.

FIG. 9: Xerogel/Aerogel (sensing component). (a) 50 μL hydrophobic alginatesodium cholate xerogels/aerogels incubated in different digestive effluents inside a polyacrylamide ultrasound phantom (third gel). (I) Incubated in pure water (MilliQ), (II) simulated gastric fluid (SGF), and (III) following incubation in Bile or SIF leading to full dissolution within 24 hours and contrast loss. This is shown in example 5a.1.

(b) Superhydrophobic silica aerogel particles (Lumira; sensing component) placed and incubated in different digestive effluents inside a polyacrylamide ultrasound phantom (third gel). Top arrow indicates top part of the well and aerogel particles floating. Bottom arrow indicates bottom of the well and sunk/contrast altered aerogel particles. (I) SGF (II) SIF, (III) Bile. A patch containing silica aerogel particles as shown in this figure is described in example 5b.

FIG. 10: Negative contrast. Patch containing gas-containing vesicles (sensing component 1; change in signal upon contact with SIF) and silica nanoparticles (sensing component 2; no change in signal; used as an internal reference signal)

(a) Patch containing a 20 wt. % Acrylamide hydrogel containing 9 wt. % SiO2 and a separately prepared 20 wt % Acrylamide hydrogel containing 33% gas-containing vesicles (Ana). Ultrasound image taken after gas-containing vesicles breakdown is visible as a black “hole” in the center of the patch, indicative of a leak. This is shown in example 3c.

(b) Ultrasound contrast before and after contact with SIF (collapse of gas-containing vesicles). Patch containing 20 wt. % acrylamide hydrogel containing 2 wt. % silica nanoparticles (top part of the layer), a 20 wt. % acrylamide hydrogel without sensing components (middle part of the layer and a 20 wt. % acrylamide hydrogel containing 33% Ana gas vesicles (bottom part of the layer). Distance measurements were performed with the Clarius Ultrasound App. (left) Digested gas-containing vesicles show no contrast. This is shown in example 3b.

(right) Untreated sample where all gas-containing vesicles are still intact and give a contrast spatially discernible from the silica contrast.

FIG. 11: Ultrasound images of patches detecting a digestive leak. (a) Disk shaped patch containing a first gel without sensing component (50 wt % PAMPS), 2 wt % NaHCO3 (sensing component) embedded in a second gel (2 wt % agar). Condition (I) the patch is attached to a porcine intestinal tissue containing SGF (simulated gastric fluid pH=2) but the system contains no perforation. (II) the system is perforated at the level of the patch, inducing production of CO2 within the patch and a change in observable contrast (in 15 min post contact).

FIG. 12: Ultrasound images performed using a Clarius 7HD probe controlled by an Ipad, performed at discrete time intervals of a 2 wt % agar gel (diameter: 8 mm, thickness: 2 mm; not encapsulated in a further gel) containing 2 wt % of sodium bicarbonate incubated in simulated gastric fluid (SGF), simulating gastric leak and monitored at discrete time intervals. The agar disk was soaked in PBS prior to application. Strong contrast increase at 15 minutes up to 4 hours. A patch containing 2 wt % of sodium bicarbonate in a 2 wt % agal gel is described in example 4a.

FIG. 13: Ultrasound images performed using a Clarius 7HD probe controlled by an Ipad, performed at discrete time intervals of a patch containing a first gel without sensing component (PAAm 20 wt %) and a second gel (2 wt % agar gel disk) containing 2 wt % sodium bicarbonate. The agar gel disk (diameter: 8 mm, thickness: 2 mm) was semi-encapsulated in the first gel. This is shown in example 4b. The patch was soaked in PBS prior to application. The patch was incubated in simulated gastric fluid (SGF), simulating gastric leak and monitored at discrete time intervals.

FIG. 14: Ultrasound images performed using a Clarius 7HD probe controlled by an Ipad, performed at discrete time intervals of a patch containing a first gel without sensing component (PAAm 20 wt %) and a second gel (2 wt % agar gel disk) containing 2 wt % sodium bicarbonate. The agar gel disk (diameter: 8 mm, thickness: 2 mm) was fully encapsulated in the first gel. This is shown in example 4c. The patch was soaked in PBS prior to application. The patch was incubated in simulated gastric fluid (SGF), simulating gastric leak and monitored at discrete time intervals. Signal stable for more than 24 hours.

FIG. 15: X-ray CT sensing components.

(a) (Left): poly(2-acrylamido-2-methyl-1-propanesulfonic acid) (PAMPS; first gel) containing 5 wt. % tantalum oxide (sensing component) before exposure to SIF. This is shown in example 9a.

(Right): PAMPS containing 5 wt. % tantalum oxide (sensing component) after exposure to SIF. Strong swelling and contrast change detectable.

(b) (Left): PNAGA (second gel) containing 10 wt. % tantalum oxide (sensing component), as described in example 9b, before exposure to SIF. (Right): PNAGA containing 10 wt. % tantalum oxide after exposure to SIF only shows minimal swelling and no discernible contrast change. This indicates that a patch (cf. FIG. 16) comprising both a first gel with a relatively higher swelling ratio and second gel with a relatively lower swelling ratio, both containing the same sensing component, gives a good contrast under CT (second gel serves as a reference signal for the change in contrast observed in the first gel).

For both a) and b), CT scan was performed under a constant angle acquisition using a single X-Ray source and a KVP of 120 kV. The images were reconstructed using a UR77 kernal.

FIG. 16: (a) Cylindrical patch and (b) Rectangular patch under CT.

Patch containing a first gel (PAMPS; top) containing 5 wt. % tantalum oxide (sensing component) and a second gel (PNAGA, bottom) containing 5 wt. % tantalum oxide (sensing component) before exposure to SIF (left image of a) and b) and after exposure to SIF (right image of a) and b). Patches shown in this figure are a combination of the individual gels shown in FIG. 15 and are described in example 10 (a) and (b). For both a) and b), CT scan was performed under a constant angle acquisition using a single X-Ray source and a KVP of 120 kV. The images were reconstructed using a UR77 kernal.

FIG. 17: Dual contrast sensing components. (a) CT and ultrasound images of 2 wt. % Agar (low swelling) containing 35 wt. % NaHCO₃.

(Two images on the left): CT and ultrasound image after SGF (+10% HCl) exposure. Resulting bubble structure is visible under CT and ultrasound.

(Two images on the right): CT and ultrasound image of sensing components before SGF exposure. Gel composition described in example 4d.

b) CT and ultrasound images of 2 wt. % Agar (low swelling) containing 35 wt. % CaCO3

(Two images on the left): CT and ultrasound image after SGF (+10% HCl) exposure. CO2 bubbles are visible in ultrasound and a decreased contrast is seen in CT.

(Two images on the right): CT and ultrasound image of gels before SGF exposure. Gel composition described in example 4e.

For both a) and b), CT scan was performed under a constant angle acquisition using a single X-Ray source and a KVP of 120 kV. The images were reconstructed using a UR77 kernal.

Ultrasound images were obtained with a Clarius L7HD probe controlled by an iPad. Samples were imaged in a 20 wt. % acrylamide hydrogel sample holder with PBS as an acoustic coupler.

FIG. 18: CT and ultrasound images of a patch containing a first gel (PAAm, high swelling) containing 40% gas vesicles (sensing component 1) and a second gel (PNAGA, low swelling) containing 5 wt % tantalum oxide (sensing component 2).

(Two images on the left): CT and ultrasound image of patches after incubation in SIF over night. Collapsed gas-containing vesicles give no contrast under ultrasound.

(Two images on the right): CT and ultrasound image of patches prior to SIF exposure. Gas-containing vesicles give a clear contrast under ultrasound.

(a) Dog bone rendition of the patch. This is shown in example 11a.

(b) Rectangular patch. This is shown in example 11b.

FIG. 19: Rectangular layered patches. (I) Patch containing a layer containing a third gel (PAMPS 50 wt %) containing alginate-cholate aerogel particles (50 μL) and a backing layer containing a polyurethane hydrogel (Hydromed D4). This is shown in example 5a.2. (II) Patch containing a layer containing a third gel (PAMPS 50 wt %) containing commercially available superhydrophobic silica aerogel particles (lumira) and a backing layer containing a polyurethane hydrogel (Hydromed D4). This is shown in example 5b.

Surprisingly, it has been found that a patch comprising:
i) a layer comprising
one or more first portions containing a first gel;
one or more second portions containing a second gel; and
at least one sensing component embedded within the first gel, the second gel, or the first gel and the second gel;
the at least one sensing component being selected from gas-containing vesicles, gas-containing microbubbles, salt containing particles, metal or metal oxide nanoparticles, carbide nanoparticles, radio-opaque iodinated or brominated organic compounds, gadolinium complexes, monosaccharide containing microspheres, polysaccharide containing protein microspheres, containing microspheres, poly(vinylpyrrolidone)-iodine complex particles, and combinations thereof;
the first gel and the second gel being selected from the group consisting of a hydrogel, an aerogel, and a xerogel, and comprising a synthetic polymer, a natural polymer, or a mixture thereof; and
wherein
i-1) the first gel is non-degradable and the second gel is degradable, or
i-2) the first gel and the second gel are non-degradable, the first gel has a relative swelling ratio, and the second gel has a relative swelling ratio, and the relative swelling ratio of the first gel is different from the relative swelling ratio of the second gel, with the relative swelling ratio being the ratio between the mass of said gel after incubation for 24 h at 37° C. in a simulated intestinal fluid and the mass of said gel prior to incubation in the simulated intestinal fluid; or
ii) a layer containing a third gel comprising a synthetic polymer, a natural polymer, or a mixture thereof, and being a hydrogel, and
at least one sensing component embedded within said third gel,
the at least one sensing component being selected from the group consisting of aerogel particles, and xerogel particles, wherein
ii-1) the at least one sensing component is degradable and the third gel is nondegradable,
ii-2) the at least one sensing component is non-degradable, the third gel is non-degradable, the at least one sensing component has a relative swelling ratio and the third gel has a relative swelling ratio, and the relative swelling ratio of the sensing component is different from the relative swelling ratio of the third gel, with the relative swelling ratio being the ratio between the mass of said at least one sensing component, or of said third gel after incubation for 24 h at 37° C. in a simulated intestinal fluid and the mass of said at least one sensing component, or of said hydrogel prior to incubation in the simulated intestinal fluid, or
ii-3) the at least one sensing component is non-degradable and the third gel is degradable; enables the detection by at least one non-invasive imaging technique a leak of a biological fluid at an application site, such as a suture site, a stapled site or an implantation site of a surgical implant.

As used herein, the term “biological fluid” includes, but it is not limited to, bile, pancreas fluid, gastric fluid, intestinal fluid, colon content, fluids containing bacteria and bacteria products, and uterus fluids.

The at least one non-invasive imaging technique is preferably selected from ultrasound imaging, computer tomography, magnetic resonance imaging, magnetic particle imaging and radio-frequency identification, more preferably from ultrasound imaging, computer tomography, magnetic resonance imaging, and radio-frequency identification, most preferably from ultrasound imaging, and computer tomography.

The patch claimed and described herein may have any shape and size suitable to be applied on a suture site, a stapled site or an implantation site and may contain additional layers, such as a backing layer, or a hydrogel support layer. Particularly suitable backing layers are described in the pending European patent application no. 21152240.4. Particularly suitable hydrogel support layers are described in the pending European patent application no. 21152240.4. The skilled person, taking into consideration the common technical knowledge in the medical field, would know and/or select the appropriate shape and size of the patch in light of the disease/condition for which said patch is used. For example, a patch with a cylindrical shape, a thickness of 2-10 mm, a length of 30-100 mm a diameter of 20-50 mm is suitable for e.g. for detecting a leak of a biological fluid at sutured or stapled regions of the small or large intestine. For example, a patch with a rectangular shape, a thickness of 1-30 mm, a length of 40-200 mm and a width of 10-30 mm is suitable for e.g. detecting a leak of a biological fluid at sutured or stapled regions of the small or large intestine or oesophageal resection closing sutures or staples. For example, a patch with a ring shape, a thickness of 1-10 mm, an inner diameter of 10-50 mm and an outer diameter of 50-100 with is suitable for e.g. for detecting a leakage of a biological fluid at a portacaval anastomosis site. For example, a patch with a triangular shape, a thickness of 1-15 mm, and a side length of 50-100 mm is suitable for e.g. for detecting a leakage of a biological fluid at biliary diversion reconnections sites. The patch may be also provided with a rectangular shape that can be cut by the surgeon to the appropriate shape and size, for example with a length of about 1-15 cm, such as about 3, 4.8, 5, 9.5 or 10 cm, and with a width of about 1-10 cm, such as about 2, 2.5, 4.8 or 5 cm.

As used herein the term “layer” refers to a 3D structure (i.e. a structure having three different coordinates: x, y, z) having a thickness (z coordinate) significantly lower than the others two dimensions (x and y coordinates). The surface (i.e. the 2D structure having the x and y coordinates) of the layer may have any shape and size suitable to be applied on a suture site, a stapled site or an implantation site of a surgical implant, including a disk shape, a rectangular shape, a rhombohedral shape, a trapezoidal shape, a ring shape, and a triangular shape. The layer described herein may have a thickness of 0.5-40 mm. The dimensions of the surface of the layer are highly dependent on the dimensions of the suture site, stapled site or implantation site of the surgical implant. The width of the layer described herein may be of 5 mm to 70 mm. The length of the layer described herein may be of 5 mm to 400 mm.

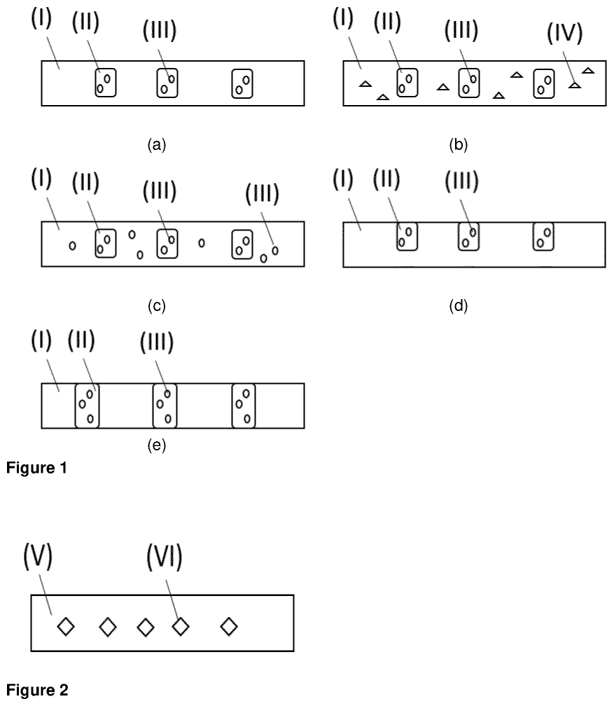

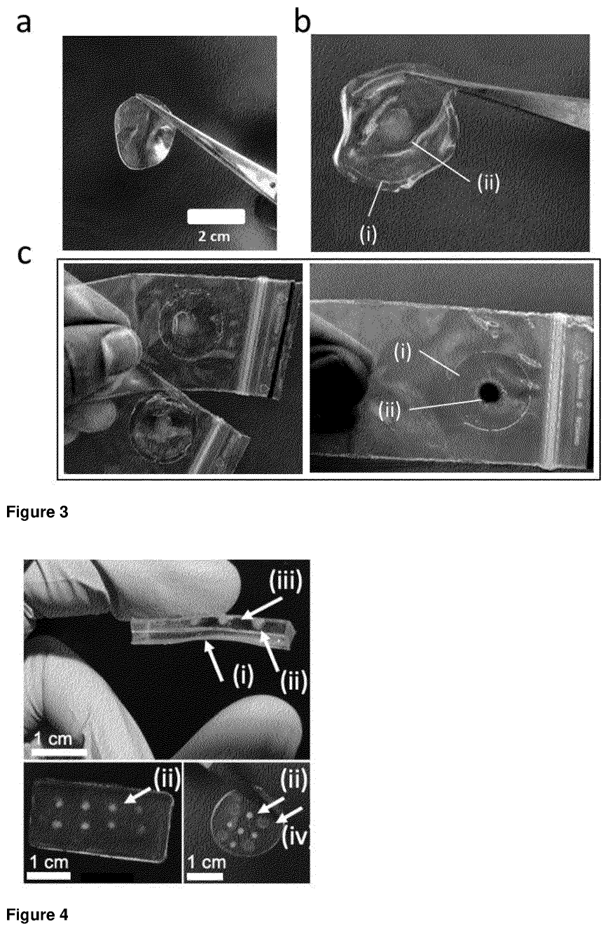

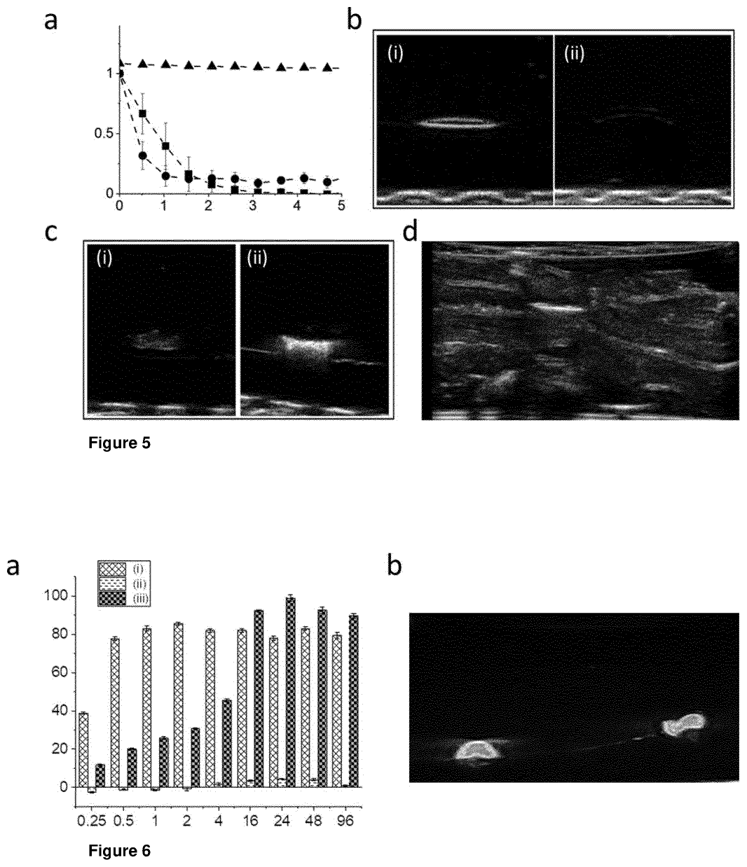

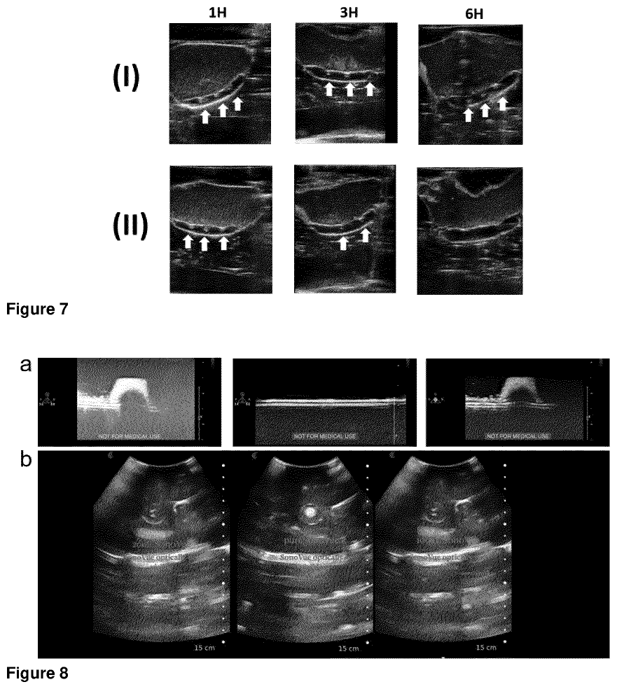

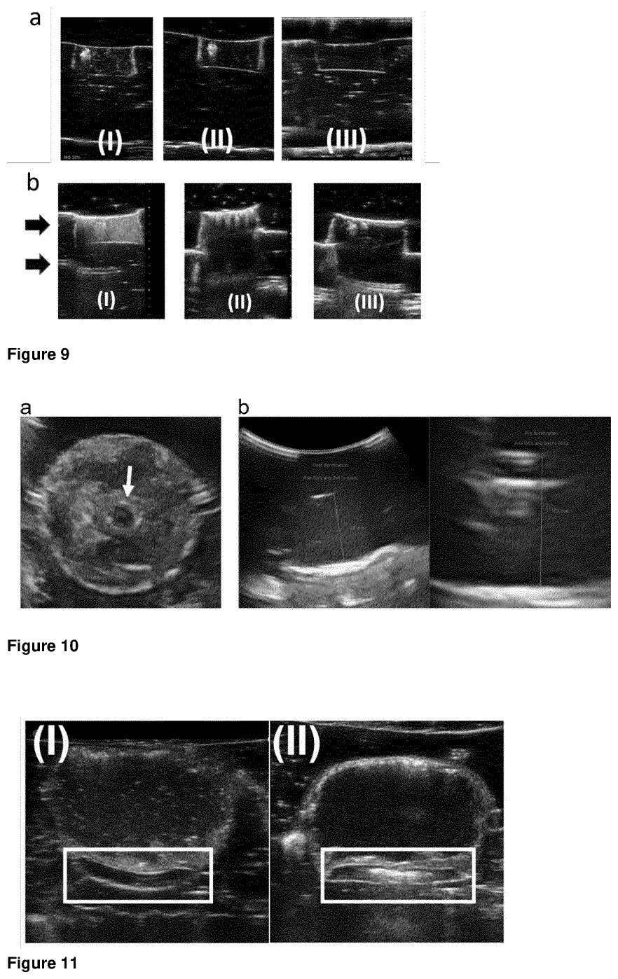

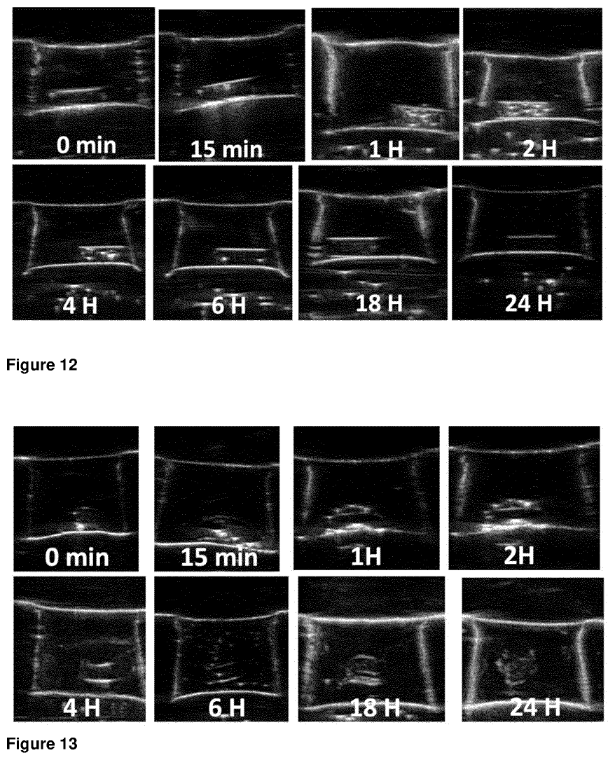

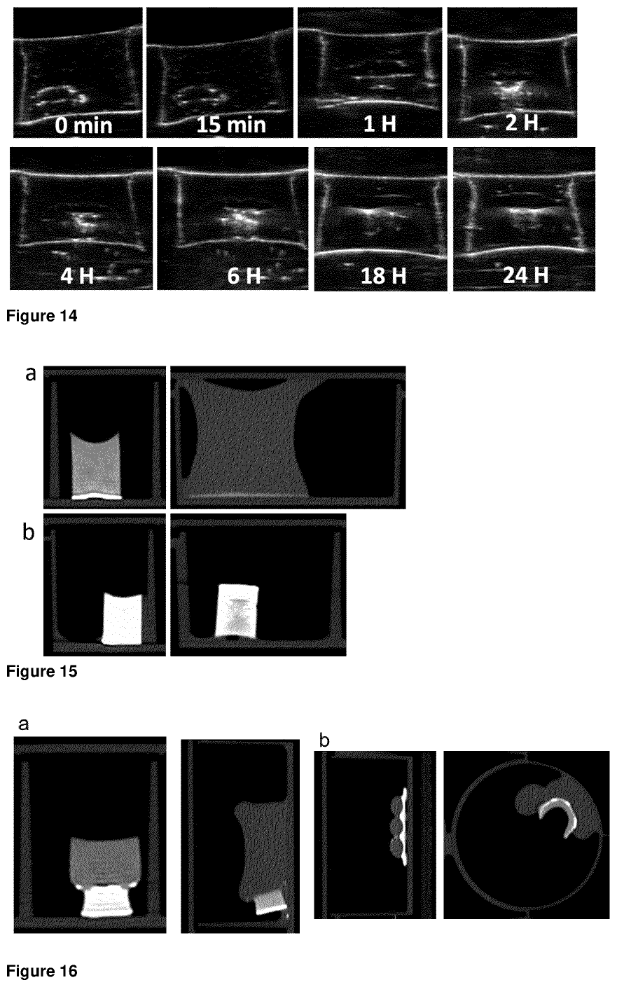

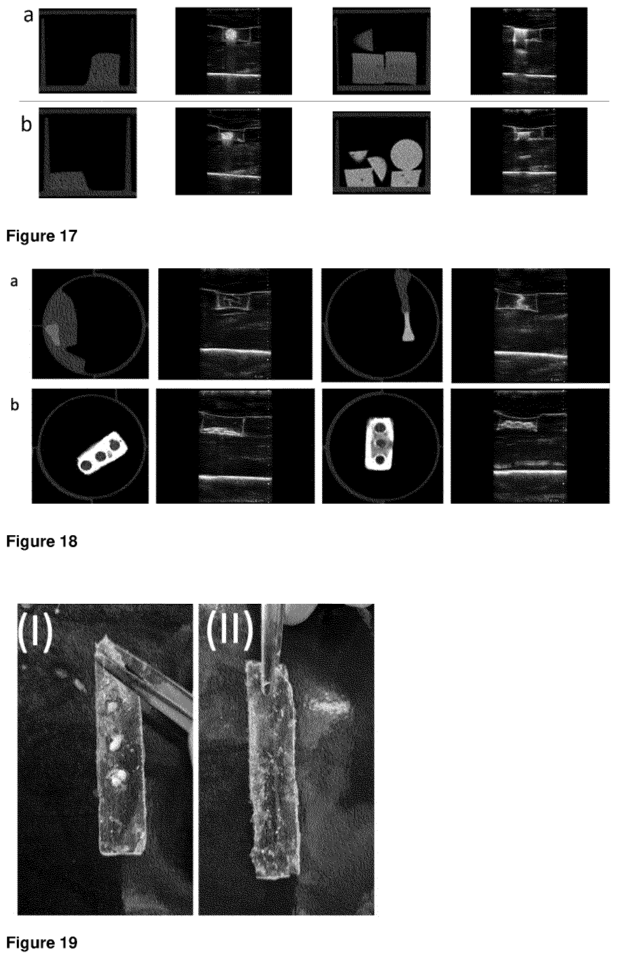

As used herein, the terms “one or more first portions” and “one or more second portions” refer to distinct 3D structures (i.e. structures having three different coordinates: x, y, z and a volume) within the layer (i.e. a substructure of said layer) having a thickness (z coordinate) equal to the thickness of the layer. The first portions and the second portions may be adjacent to each other as shown for example in FIG. 1. The first portions may be, optionally uniformly, distributed within a continuous second portion or the second portions may be, optionally uniformly, distributed within a continuous second portion. Besides the “one or more first portions” and “one or more second portions”, the layer may contain further portions containing either a gel that is different from the gel contained by the first portions and/or second portions, or the gel contained by the first portions or the second portions having embedded therein a contrast agent, such as silica powder, and hydroxyapatite. The first portions contain a first gel as described herein, which preferably occupies at least 70%, more preferably at least 80%, mostly preferably at least 90% of the volume of said first portions. The second portions contain a second gel as described herein, which occupies at least 50%, more preferably at least 70%, most preferably at least 80% of the volume of the second portions.

As used herein, the expression “at least one sensing component” includes one sensing component, or two or more different sensing components.

As used herein, the expression “at least one sensing component embedded within said first gel/second gel/hydrogel” means that the at least one sensing component is enclosed within said first gel/second gel/hydrogel. The at least one sensing is preferably uniformly dispersed within the entire volume of said first gel, second gel, and hydrogel, respectively.

The at least one sensing component may be embedded within the first gel, the second gel, or the first gel and the second gel. Preferably, the at least one sensing component is embedded both in the first gel and the second gel (see FIG. 1(b) and FIG. 1(c)). The sensing component(s) present in the first gel and the second gel may be the same (see FIG. 1(c)) (e.g.: barium sulphate particles, or tantalum oxide nanoparticles embedded both in the first gel and the second gel), or may be different (see FIG. 1(b)) (e.g.: one gel encloses gas-containing vesicles and the other gel encloses tantalum oxide nanoparticles).

The term “relative swelling ratio” as used in the context of the present invention is the ratio between the mass of a gel (e.g. as an aerogel, a xerogel, or of particles (e.g. aerogel particles, xerogel particles)) after incubation for 24 h at 37° C. in simulated intestinal fluid and the mass of said gel or particles prior to incubation in the simulated intestinal fluid.

As used herein, the term “simulated intestinal fluid” refers to a fluid obtained as described in the Test Solutions of the United States Pharmacopeia 30, NF 25, 2007.

The patch claimed and described herein enables monitoring of the application site and thereby, alleviating the need for artificial drains to the patient's body. Further, the patch claimed and described herein mitigates the risk for septic peritonitis or other adverse events in the case of leakage of a sutured or stapled soft tissue reconnection site due to early detection of said leakage.

The terms “monitoring” and “monitor” as used in the context of the present invention relate to the detection of a leak formation at a suture site, stapled site, or an implantation site of a surgical implant, in particular in the abdominal region, more particularly an anastomosis site. Upon contact with a biological fluid, the at least one sensing component embedded within the first gel and/or the second gel may be transformed/activated or released, leading to a detectable contrast signal between the one or more first portions and the one or more second portions of the layer. Alternatively, upon contact with a biological fluid the aerogel/xerogel particles swelling at a lower swelling rate than the hydrogel surrounding them provide a contrast signal between aerogel/xerogel particles and the hydrogel.

“Transformation/activation” of a sensing component refers for example to the collapse of a gas-containing vesicles, or the generation of a detectable component, such as the generation of Fe³⁺ as a dissolution product of Fe₂O₃, or the generation of CO₂ from carbonates (e.g. sodium bicarbonate) upon contact with a biological fluid.

In certain embodiments, the at least one sensing component may be released from the layer upon contact with a biological fluid. The release of the at least one sensing component may occur due to the swelling of the gel embedding the at least one sensing component, or the cleavage of the at least one sensing component from the synthetic polymer and/or the natural polymer (full or partial degradation of said polymer).

Independently of each other, the first gel, the second gel and the third gel comprises a synthetic polymer, a natural polymer, or a mixture thereof that can be degradable or non-degradable.

Preferably, the synthetic polymer described herein is a polymer having monomeric units selected from the group consisting of methacrylates, acrylates, vinyls, thiols, polyurethane forming monomers, and mixtures thereof.

Suitable methacrylates may be selected from the group consisting of methacrylic acid (MA), methyl methacrylate, methacrylamide, hydroxyethylmethacrylate, ethyl hexyl (meth)acrylate, glycidyl methacrylate, oligoethylene-glycol methylacrylate, 2-(dimethylamino)ethyl methacrylate, [2-(methacryloyloxy)ethyl] trimethylammonium chloride, N-(2-hydroxypropyl) methacrylamide, methacrylic anhydride, N,N-diethylmethacrylamide, (hydroxyphenyl) methacrylamide, 2-hydroxypropyl methacrylamide, 2-aminoethylmethacrylamide hydrochloride, methacryloyl-L-Lysine, phosphoric acid 2-hydroxyethyl acrylate ester, 4-methacryloxyethyl trimellitic anhydride, 3-sulfopropyldimethyl-3-methacrylamidopropylammonium, methacryloyloxyethyl phosphorylcholine (MPC), 2-N-morpholinoethyl methacrylate, 2-aminoethyl methacrylate hydrochloride, methacryloyl-L-lysine, pyridyl disulfide ethyl methacrylate, N-(3-aminopropyl) methacrylamide hydrochloride, N-(3-BOC-aminopropyl) methacrylamide, O-nitrobenzyl methacrylate, O-nitrobenzyl ethyl methacrylate. It is also envisaged to use combinations of two or more methacrylates.

Preferably, methacrylates are selected from the group consisting of methacrylic acid, methyl (methacrylate), methacrylamide, hydroxyethylmethacrylate, ethyl hexyl (meth)acrylate, glycidyl methacrylate, oligoethylene-glycol methylacrylate, 2-(dimethylamino)ethyl methacrylate, [2-(methacryloyloxy)ethyl] trimethylammonium chloride, N-(2-hydroxypropyl) methacrylamide, methacrylic anhydride, N,N-diethylmethacrylamide, 2-hydroxypropyl methacrylamide, methacryloyloxyethyl phosphorylcholine (MPC), 2-aminoethyl methacrylate hydrochloride, pyridyl disulfide ethyl methacrylate, O-nitrobenzyl methacrylate and O-nitrobenzyl ethyl methacrylate, and mixtures thereof.

Suitable acrylates may be selected from the group consisting of acrylic acid (AA), acrylamide (AAm), hydroxyethylacrylate, ethyl hexyl acrylate, butyl acrylate, 2-(dimethylamino)ethyl acrylate, (acrylamidopropyl)trimethylammonium chloride, N-(2-hydroxypropyl) acrylamide, N-acryloyl glycinamide, acrylated adenine, acrylated thymine, acrylated cytosine, acrylated guanine, acrylated uracyl, NHS acrylate, NHS sulfo acrylate, acrylic anhydride, 2-acrylamido-2-methyl-1-propanesulfonic, N,N-diethylacrylamide, (hydroxyphenyl) acrylamide, 2-hydroxypropyl acrylamide, Nisopropylacrylamide, beta-carboxyethyl acrylate, 2-N-morpholinoethyl acrylate, 2-aminoethyl acrylate hydrochloride, N-[3-(N,N-dimethylamino) propyl] acrylamide, 2-acryloxyethyltrimethylammonium chloride, 2-cyanoethyl N-acrylate, acryloxysuccinimide, O-nitrobenzyl acrylate, and O-nitrobenzyl ethyl acrylate. It is also envisaged to use combinations of two or more acrylates.

Preferably, acrylates are selected from the group consisting of acrylic acid, acrylamide, hydroxyethylacrylate, ethyl hexyl acrylate, butyl acrylate, 2-(dimethylamino)ethyl acrylate, (acrylamidopropyl)trimethylammonium chloride, N-(2-hydroxypropyl) acrylamide, N-acryloyl glycinamide, NHS acrylate, NHS sulfo acrylate, acrylic anhydride, N,N-diethylacrylamide, (hydroxyphenyl) acrylamide, 2-hydroxypropyl acrylamide, N-isopropylacrylamide, beta-carboxyethyl acrylate, 2-aminoethyl acrylate hydrochloride, N-[3-(N,N-dimethylamino) propyl] acrylamide, 2-acryloxyethyltrimethylammonium chloride, 2-cyanoethyl N-acrylate, acryloxysuccinimide, O-nitrobenzyl acrylate, and O-nitrobenzyl ethyl acrylate, and mixtures thereof.

Suitable vinyls may be selected from the group consisting of vinyl pyrrolidinone, vinylcaprolactam, sodium styrene sulfonate, 2-methylene-1,3-dioxepane, 3-(acrylamido)phenylboronic acid, allyl methyl sulfone, 3-allyloxy-2-hydroxy-1-propanesulfonic acid sodium salt, 3-allyloxy-1,2-propanediol, N-methyl-N-vinyl acetamide, N-vinyl acetamide (NVA), vinylphosphonic acid, allylphosphonic acid monoammonium salt, mutliarm PEG (2 or 3 or 4 arms) and vinyl sulfone. It is also envisaged to use combinations of two or more vinyls.

Preferably, vinyls are selected from the group consisting of vinyl pyrrolidinone, vinylcaprolactam, sodium styrene sulfonate, and mixtures thereof.

Suitable thiols may be selected from the group consisting of 1,4-butanedithiol, 1,3-propanedithiol, 1,2-ethanedithiol, 2,2′ (ethylenedioxy) diethanethiol, ethylene glycol bis-mercaptoacetate, 2-arm PEG-SH, 3-arm PEG-SH, 4-arm PEG-SH. It is also envisaged to use combinations of two or more thiols.

Preferably, thiols are selected from 1,3-propanedithiol, 2,2′ (ethylenedioxy) diethanethiol, and mixtures thereof.

Polyurethanes are formed by reactions of polyols, such as 2,4,6-tria-mino-1,3,5-triazine, 3,5-dimethoxy-4-hydroxybenzaldehyde, polyethylene glycol (PEG 400, 600, 1000), 1,4-butanediol, and CAPA™3050, and isocyanates, such as Hexamethylene diisocyanate, sophoronediisocyanat, L-lysine ethyl ester diisocyanate, isophorone diisocyanate. Two or more polyols and two or more isocyanates may be used to form the synthetic polymer.

Commercially available polyurethanes include, but are not limited to, Hydromed D1, Hydromed D2, Hydromed D3, Hydromed D4, Hydromed D6, Hydromed D640, Hydromed D7 and HydroSlip C.

Typically, the synthetic polymer contains one or more monomeric units comprised functioning as crosslinkers. Incorporation of crosslinkers into the synthetic polymer enables the formation of a 3D-network, such as a hydrogel. Such crosslinkers are known in the field and selected in accordance with the monomer units of the synthetic polymer.

Some monomers, particularly monomers that have relatively strong H-bond interactions, such as N-acryloyl glycinamide (NAGA) do not require the addition of crosslinkers to form a hydrogel. However, most monomers require addition of crosslinkers to form a hydrogel.

As convenient in the field, polymers may comprise different classes of monomers as mentioned above, e.g. copolymers of acrylates and methacrylates.

Suitable crosslinkers may be selected from the group consisting of polyethylene glycol dimethacrylate (particularly PEGDMA 20000), 1,6-hexanediol diacrylate, 1,4-butanediol diacrylate, 1,4-phenylene diacrylate, bis(2-methacryloxyethyl)phosphate, triethylene glycol diacrylate (TriEGDA), dipentaerythritol pentaacrylate, 1,1,1-trimethylolpropane triacrylate (TriMPTA), 1,1,1-trimethylolpropane trimethacrylate, PEO (5800)-b-PPO (3000)-b-PEO (5800)dimethacrylate, pentaerythritol triacrylate, pentaerythritol tetraacrylate, tricyclodecane dimethanol diacrylate, PEGDA-PEG diacrylate, zinc dimethacrylate, carboxybetaine disulfide cross-linker (CBX-SS), oxidized alginate, diselenide crosslinker, 2,2-dimethacroyloxy-1-ethoxypropane (DMAEP), N,N′-bis(acryloyl) cystamine (BAC), methylene bis-acrylamide (mBAA), 1,3,5-triallyl-1,3,5-triazine-2,4,6 (1H,3H,5H)-trione, mutliarm PEG (2,3,4)thiol terminated, multiarm PEG (2 or 3 or 4 arms) vinyl sulfone terminated, multiarm PEG (2,3,4) NHS terminated, multiarm PEG (2 or 3 or 4 arms) NH2 terminated, 2,4,6-triamino-1,3,5-triazine, 3,5-dimethoxy-4-hydroxybenzaldehyde, polyethylene glycol (PEG 400, 600, 1000).

Preferably, the crosslinkers contained by the synthetic polymer described herein, are selected from the group consisting of polyethylene glycol dimethacrylate (PEGDMA 20000), 1,4-butanediol diacrylate, pentaerythritol triacrylate, PEGDA-PEG diacrylate, zinc dimethacrylate, carboxybetaine disulfide cross-linker (CBX-SS), oxidized alginate, 2,2-dimethacroyloxy-1-ethoxypropane (DMAEP), N, N′-Bis(acryloyl) cystamine (BAC), methylene bis-acrylamide, multiarm PEG (2 or 3 or 4 arms) vinyl sulfone terminated, and multiarm PEG (2 or 3 or 4 arms) NHS terminated, and mixtures thereof.

Particularly preferred synthetic polymers are selected from polyacrylamide, poly(2-acrylamido-2-methyl-1-propanesulfonic acid), poly(N-(2-hydroxyethyl) acrylamide) (PNHEA), poly(acrylamide-co-methyl acrylate-co-acrylic acid), poly(methylacrylate-co-acrylic acid) and poly(N-acryloyl glycinamide) (PNAGA).

As used herein, the term “natural polymer” encompasses purely natural polymer such as gelatin, alginate, chitosan, dextran, heparin, hyaluronic acid, gum arabic, kappa-carraggennans, cellulose, agar, starch, such as potato starch, psyllium husk starch and Eurylon7, pectin, such as pectin from citrus peel, xanthum gum, guar gum, gellan gum, bovine serum albumin, and human serum albumin, and chemically modified natural polymers, such as cholic-acid-alginate, carboxymethylcellulose, hydroxypropyl methylcellulose, acrylated elastin, acrylated albumin, acrylated alginate, and gluteraldehyde crosslinked chitosan. The natural polymers described herein may be degradable (e.g.: bovine serum albumin, human serum albumin, agar, cholic acid-alginate), or non-degradable (e.g.: cellulose, such as cellulose nanofibers, carboxymethylcellulose).

A first embodiment according to the present invention relates to a patch comprising a layer comprising
one or more first portions containing a first gel;
one or more second portions containing a second gel; and
at least one sensing component embedded within the first gel, the second gel, or the first gel and the second gel;
the at least one sensing component being selected from gas-containing vesicles, gas-containing microbubbles, salt containing particles, metal or metal oxide nanoparticles, carbide nanoparticles, radio-opaque iodinated or brominated organic compounds, gadolinium complexes, monosaccharide containing microspheres, polysaccharide containing protein microspheres, containing microspheres, poly(vinylpyrrolidone)-iodine complex particles, and combinations thereof;
the first gel and the second gel being selected from the group consisting of a hydrogel, an aerogel, and a xerogel, and comprising a synthetic polymer, a natural polymer, or a mixture thereof; and
wherein the first gel is non-degradable and the second gel is degradable. The non-degradable first gel comprises preferably one or more of the synthetic polymers described herein and/or one or more of the non-degradable natural polymers described herein, such as cellulose or carboxymethylcellulose, more preferably one or more of the synthetic polymers described herein having monomeric units selected from the group consisting of acrylamide, acrylic acid, methyl acrylate, 2-hydroxyethyl acrylate, 2-hydroxyethyl acrylamide, sodium 2-acrylamido-2-methylpropane sulfonate, N-acryloyl glycinamide, styrene sulfonate, m-PEG acrylate (30′000), N-tris(hydroxymethyl) methyl acrylamide, bis-acrylamide, polyethylene glycol diacrylate, N, N′-bis(acryloyl) cystamine, and mixtures thereof, most preferably one or more synthetic polymers selected from polyacrylamide, poly(2-acrylamido-2-methyl-1-propanesulfonic acid), poly(N-(2-hydroxyethyl) acrylamide) (PNHEA), poly(acrylamide-co-methyl acrylate-co-acrylic acid), and poly(methylacrylate-co-acrylic acid. The degradable second gel comprises preferably a degradable natural polymer, such as bovine serum albumin, human serum albumin, agar, or alginate, cholic acid-alginate, or a degradable synthetic polymer having a crosslinked network, such as PNHEA-BAC.

A second embodiment according to the present invention relates to a patch comprising a layer comprising
one or more first portions containing a first gel;
one or more second portions containing a second gel; and
at least one sensing component embedded within the first gel, the second gel, or the first gel and the second gel;
the at least one sensing component being selected from gas-containing vesicles, gas-containing microbubbles, salt containing particles, metal or metal oxide nanoparticles, carbide nanoparticles, radio-opaque iodinated or brominated organic compounds, gadolinium complexes, monosaccharide containing microspheres, polysaccharide containing microspheres, protein containing microspheres, poly(vinylpyrrolidone)-iodine complex particles, and combinations thereof;
the first gel and the second gel being selected from the group consisting of a hydrogel, an aerogel, and a xerogel, and comprising a synthetic polymer, a natural polymer, or a mixture thereof; and
wherein the first gel and the second gel are non-degradable, the first gel has a relative swelling ratio, and the second gel has a relative swelling ratio, and the relative swelling ratio of the first gel is different from the relative swelling ratio of the second gel, with the relative swelling ratio being the ratio between the mass of said gel after incubation for 24 h at 37° C. in a simulated intestinal fluid and the mass of said gel prior to incubation in the simulated intestinal fluid. Preferably, there is a difference of at least 3 in absolute value between the relative swelling ratio of the first gel and the relative swelling ratio of the second gel. More preferably, there is a difference of at least 4, such as at least 5, at least 10, at least 15, or at least 20, in absolute value between the relative swelling ratio of the first gel and the relative swelling ratio of the second gel. The non-degradable first gel and the non-degradable second gel comprises preferably one or more of the synthetic polymers described herein and/or one or more of the non-degradable natural polymers described herein, such as cellulose or carboxymethylcellulose, more preferably one or more of the synthetic polymers described herein having monomeric units selected from the group consisting of acrylamide, acrylic acid, methyl acrylate, 2-hydroxyethyl acrylate, 2-hydroxyethyl acrylamide, sodium 2-acrylamido-2-methylpropane sulfonate, Nacryloyl glycinamide, styrene sulfonate, N-tris(hydroxymethyl)methyl acrylamide, bisacrylamide, polyethylene glycol diacrylate, N,N′-bis(acryloyl) cystamine, and mixtures thereof. Most preferably, the non-degradable first gel comprises one or more synthetic polymers selected from PAMPS, PHNEA, P (AA-MA), P (AAm-MA-AA), and PAAm. Most preferably, the non-degradable second gel comprises one or more of the synthetic polymers selected from PNAGA, PAAm, Hydromed D1, Hydromed D2, Hydromed D3, Hydromed D4, Hydromed D6, Hydromed D640, Hydromed D7 and HydroSlip C. The non-degradable first gel and the non-degradable second gel are to be chosen so that their relative swelling ratios are different. A difference of at least 3, preferably of at least 4, such as of at least 5, 10, 15, or 20, in absolute value between the relative swelling ratio of the first gel and the relative swelling ratio of the second gel is preferred.

A third embodiment according to the present invention relates to a patch comprising a layer containing a third gel comprising a synthetic polymer, a natural polymer, or a mixture thereof, and being a hydrogel, and
at least one sensing component embedded within said third gel,
the at least one sensing component being selected from the group consisting of aerogel particles, and xerogel particles, wherein the at least one sensing component is degradable and the third gel is a non-degradable. The degradable sensing component selected from aerogel particles, and xerogel particles preferably contains a degradable natural polymer, such as alginate, including cholic acid-alginate, bovine serum albumin, and human serum albumin. The non-degradable third gel preferably comprises one or more of the synthetic polymers described herein and/or one or more of the non-degradable natural polymers described herein, such as cellulose, cellulose nanofibers or carboxymethylcellulose, more preferably one or more synthetic polymers having monomeric units selected from the group consisting of acrylamide, acrylic acid, methyl acrylate, 2-hydroxyethyl acrylate, 2-hydroxyethyl acrylamide, sodium 2-acrylamido-2-methylpropane sulfonate, N-acryloyl glycinamide, styrene sulfonate, N-tris(hydroxymethyl)methyl acrylamide, bis-acrylamide, polyethylene glycol diacrylate, N,N′-bis(acryloyl) cystamine, and mixtures thereof, most preferably one or more polymers selected from PAMPS, PHNEA, P (AA-MA), P (AAmMA-AA), and PAAm.

A fourth embodiment according to the present invention relates to a patch comprising a layer containing a third gel comprising a synthetic polymer, a natural polymer, or a mixture thereof, and being a hydrogel, and at least one sensing component embedded within said third gel, wherein
the at least one sensing component being selected from the group consisting of aerogel particles, and xerogel particles,
the at least one sensing component is non-degradable,
the third gel is non-degradable,
the at least one sensing component has a relative swelling ratio and the third gel has a relative swelling ratio, and the relative swelling ratio of the sensing component is different from the relative swelling ratio of the third gel, with the relative swelling ratio being the ratio between the mass of said at least one sensing component, or of said third gel after incubation for 24 h at 37° C. in a simulated intestinal fluid and the mass of said at least one sensing component, or of said hydrogel prior to incubation in the simulated intestinal fluid. Preferably, there is a difference of at least 3 in absolute value between the relative swelling ratio of the sensing component and the relative swelling ratio of the third gel. More preferably, there is a difference of at least 4, such as at least 5, at least 10, at least 15, or at least 20, in absolute value between the relative swelling ratio of the sensing component and the relative swelling ratio of the third gel. Examples of non-degradable aerogel particles, and xerogel particles include hydrophobic millimeter-sized particles of silica aerogel commercialized under the name Lumira® and glutaraldehyde crosslinked chitosan. The nondegradable third gel preferably comprises one or more of the synthetic polymers described herein and/or one or more of the non-degradable natural polymers described herein, such as cellulose or carboxymethylcellulose, more preferably one or more synthetic polymers having monomeric units selected from the group consisting of acrylamide, acrylic acid, methyl acrylate, 2-hydroxyethyl acrylate, 2-hydroxyethyl acrylamide, sodium 2-acrylamido-2-methylpropane sulfonate, N-acryloyl glycinamide, styrene sulfonate, N-tris(hydroxymethyl)methyl acrylamide, bis-acrylamide, polyethylene glycol diacrylate, N,N′-bis(acryloyl) cystamine, and mixtures thereof, most preferably one or more polymers selected from PAMPS, PHNEA, P (AA-MA), P (AAmMA-AA), and PAAm.

A fifth embodiment according to the present invention relates to a layer containing a third gel comprising a synthetic polymer, a natural polymer, or a mixture thereof, and being a hydrogel, and at least one sensing component embedded within said third gel,
the at least one sensing component being selected from the group consisting of aerogel particles, and xerogel particles, wherein the at least one sensing component is non-degradable and the third gel is degradable. Examples of non-degradable aerogel particles, and xerogel particles include hydrophobic millimeter-sized particles of silica aerogel commercialized under the name Lumira® and glutaraldehyde crosslinked chitosan. The degradable third gel comprises preferably a degradable natural polymer, such as bovine serum albumin, human serum albumin, agar, dextrane or alginate, including cholic acid-alginate, or a degradable synthetic polymer having a crosslinked network, such as PNHEA-BAC.

The non-degradable gels described herein may further comprise an additional synthetic polymer selected from polyethylene glycol, such as PEG 400, PEG 600, PEG 1000, polydopamine, polygallic acid, Eudragit S100, carbopol, polycarbonates and nylon.

The gels described herein may further contain natural and/or synthetic additives. Preferably, said additives are natural additives selected from the group consisting of capric acid, lauric acid, myristic acid, palmitic acid, palmitoleic acid, stearic acid, oleic acid, ricinoleic acid, vaccenic acid, linoleic acid, alpha-linolenic acid, arachidic acid, gadoleic acid, beeswax and gamma-linolenic acid. More preferably, said additives are natural additives selected from the group consisting of stearic acid, myristic acid, palmitic acid and beeswax.

The first gel described herein and the second gel described herein are selected from the group consisting of a hydrogel, an aerogel, and a xerogel. The first gel described herein and the second gel described herein may be of the same type or of different type. Preferably, the first gel and the second gel are of the same type (i.e. they are either hydrogels, aerogels, or xerogels).

Preferably, the first gel and the second gel are hydrogels. Also preferably, the first gel and/or the third gel is a non-degradable hydrogel, more preferably a nondegradable synthetic hydrogel, most preferably a non-degradable synthetic hydrogel comprising the synthetic polymer described herein, and especially a synthetic polymer having monomeric units selected from the group consisting of acrylamide, acrylic acid, methyl acrylate, 2-hydroxyethyl acrylate, 2-hydroxyethyl acrylamide, sodium 2-acrylamido-2-methylpropane sulfonate, N-acryloyl glycinamide, styrene sulfonate, N-tris(hydroxymethyl) methyl acrylamide, bis-acrylamide, polyethylene glycol diacrylate, N,N′-bis(acryloyl) cystamine, and mixtures thereof, such as a synthetic polymer selected from PAMPS, PHNEA, P (AA-MA), P (AAm-MA-AA), PAAm, and mixtures thereof.

The term “hydrogel” is well known in the field and includes natural hydrogels (i.e. hydrogels containing a natural polymer) and synthetic hydrogels (i.e. hydrogels containing a synthetic polymer, or a mixture of a synthetic polymer and a natural polymer). Typically, a hydrogel is a network of crosslinked polymer chains that are hydrophilic and can thus bind aqueous fluids. The synthetic hydrogels are mainly identified by the monomeric units forming the polymer that in turn can form a hydrogel upon contact with a fluid. Typically, the above fluid is an aqueous fluid, e.g. water. Typically, the hydrogel comprises 10-90% water. Preferably, the hydrogel comprises 30-80% water, more preferably, 40-60% water, such as 40%, 45%, 50%, 55% or 60% water. Although the monomeric units are no longer present in the hydrogel so formed, it is convenient in the field to still refer to the chemical class of monomeric units forming such polymer. A broad range of hydrogel forming monomeric units is known. The skilled person is in a position to select monomeric units compatible with the field of surgery.

The term “aerogel” is known in the field. An aerogel is typically obtained when the liquid present within a gel network is removed at a supercritical state of the liquid. Typically, such gel networks are hydrogels as described above (i.e. the liquid is water) or alcogels (i.e. gel networks having the composition of the hydrogels described above, wherein the liquid is an alcohol instead of water). Aerogels are generally monolithic and have a lower density than xerogels.

The term “xerogel” is known in the field. A xerogel is obtained by removing the liquid present within a gel network by evaporation, e.g. by lyophilisation. Typically, such gel networks are hydrogels as described above (i.e. the liquid is water) or alcogels (i.e. gel networks having the composition of the hydrogels described above, wherein the liquid is an alcohol instead of water).

As used herein, the term “gas-containing vesicles” refers to gas-filled protein nanostructures that are isolated from natural cyanobacterial, particularly Anabaena flosaquae (Ana) and haloarchaeal, particularly Halobacterium salinarum, host organisms (Halo) or from genetically engineered organisms, such as E. coli expressing a heterologous gas vesicle gene cluster. Such gas-containing vesicles typically have a diameter of approximately 100-900 nanometers. The hollow gas interior is enclosed by a protein shell of typically 1-5 nm shell that is permeable to gas but excludes liquid water. Owing to their physical properties, gas-containing vesicles are known to serve as highly sensitive imaging agents for ultrasound and magnetic resonance imaging (MRI).

As used herein, the term “gas-containing microbubbles” refers to microstructures having a highly elastic surrounding membrane of phospholipids, which encapsulates a gas, such as air and hexafluorosulfide (SF6). Examples of suitable commercially available gas-containing microbubbles include SonoVue® microbubbles from Bracco that contain SF6 gas encapsulated within a monolayer membrane of phospholipids and having a diameter of about 1-11 micrometres.

The gas-containing vesicles and/or gas-containing microbubbles described herein, which are embedded in the gel (and are ab initio detectable by ultrasound imaging) collapse upon contact with the biological fluid, leading to the loss of an ab initio detectable signal. The collapse of the gas-containing structures, such as “gascontaining vesicles” and “gas-containing microbubbles” leads to the loss of an ab initio detectable signal and is also referred to herein as a “turn-off” type ultrasound.

As used herein, the term “salt containing particles” encompasses particles, such as microparticles and nanoparticles, containing a radio-opaque salt, such as barium sulphate particles, and particles, such as microparticles and nanoparticles, containing a salt that generates a gas, such as carbon dioxide, hydrogen sulfide, or oxygen, upon contact with a biological fluid and/or an acid and/or hydrogen peroxide. Examples of salts that generate a gas upon contact with a biological fluid and/or an acid and/or hydrogen peroxide include, but are not limited to carbonates, such as CaCO₃, NaHCO₃, and Na₂CO₃, sulphides, such as Na₂S, NaHS, and CaS, and iron chloride. The particles containing a salt that generates a gas upon contact with a biological fluid and/or an acid and/or hydrogen peroxide may be coated with a coating, such as a beeswax coating and/or may further contain an acid which is in solid state at room temperature, such as citric acid. Examples of such particles include beeswax coated particles containing a blend of NaHCO₃ and citric acid, or Na₂CO₃ and citric acid in a 1:1 molar ratio. When a sulphide is used as a salt, the layer further contains hydrogen peroxide encapsulated particles distributed within the polymer. Upon contact with a biological fluid, carbonate (CaCO₃, NaHCO₃, and Na₂CO₃) and sulphide (as Na₂S, NaHS, and CaS) containing particles generate carbon dioxide and hydrogen sulphide, respectively, which is released within the polymer and provides a signal detectable by ultrasound that was not detectable prior to the contact. The salt containing particles described herein have preferably a primary particle size from about 5 nm to 50 μm.

As used herein, the term “metal and metal oxide nanoparticles” include metal nanoparticles and metal oxide nanoparticles that are detectable by ultrasound imaging, computer tomography, magnetic resonance imaging, and magnetic particle imaging. Examples of such metal and metal oxide nanoparticles include, but are not limited to, iron, iron oxide, zinc oxide, zinc ferrite, tantalum, tantalum oxide, bismuth, bismuth oxide, bismuth ferrite, gold, manganese oxide, terbium oxide, gadolinium oxide nanoparticles, and mixtures thereof. The metal and metal oxide nanoparticles may present magnetic properties. Example of magnetic metal and metal oxide nanoparticles include iron, iron oxide, zinc oxide, zinc ferrite, manganese oxide, terbium oxide, gadolinium oxide, cobalt ferrite nanoparticles, and mixtures thereof. Preferably, the particles have a primary particle size of between 2 nm-500 nm, with the size corresponding to their largest dimension.

As used herein, the term “magnetic carbide nanoparticles” encompasses iron carbide and iron cobalt carbide nanoparticles, and mixtures thereof. Preferably, the particles have a primary particle size of between 2 nm-500 nm, with the size corresponding to their largest dimension.

Radio-opaque iodinated or brominated organic compounds include contrast agents that are detectable by computer tomography such as, diatrizoate, metrizoate, iothalamate, ioxaglate, iopamidol, iohexol, ioxilan, iopromide, iodixanol, iobitridol, ioversol, diatrizoic acid, metrizoic acid, iodamidelotalamic acid, ioxitalamic acid, ioglicic acid, acetrizoic acid, iocarmic acid, ethiodal, diodone, metrizamide, iohexol, ioxaglic acid, iopamidol, iopromide, iotrolan, ioversol, iopentol, iodixanol, iomeprol, iobitridol, loxilan, iodoxamic acid, iotroxic acid, loglycamic acid, adipiodonelobenzamic acid, iopanoic acid, iocetamic acid, sodium iopodate, tyropanoic acid, calcium iopodate, calcium iopodate, ethyl esters of iodised fatty acids, iopydol, propyliodonelofendylate, lipiodol, and perflubron.

The term “gadolinium complexes” refers to gadolinium complexes known as MRI contrast agents. Such gadolinium complexes include, but are not limited to, GdDTPA complex, gadoterate, gadobutrol, gadoteridol, gadopentetate, gadobenate, gadopentetic acid dimeglumine, gadoxentate, gadoversetamide, gadodiamide, gadofosveset, gadocoletic acid, gadomelitol, gadomer 17, gadobenic acid, gadobutrol, gadodiamide, gadofosveset, gadopentetic acid, gadoteric acid, gadoteridol, gadoversetamide, and gadoxetic acid.

The term “monosaccharide containing microspheres” refers to spherical structures having a diameter of between 0.5 to 50 μm containing a monosaccharide, such as galactose.

The term “polysaccharide containing microspheres” refers to spherical structure shaving a diameter of between 0.5 to 50 μm containing a polysaccharide, such as dextrane.

The term “protein containing microspheres” refers to spherical structure shaving a diameter of between 0.5 to 50 μm containing a protein, such as human or animal albumin.

The terms “aerogel particles”, and “xerogel particles” encompass microparticles and nanoparticles containing an aerogel, and a xerogel, respectively. Said aerogel, and xerogel may be made of silica, cholic acid-alginate, bovine serum albumin, human serum albumin, glutaraldehyde crosslinked chitosan. Examples of such particles include hydrophobic millimeter-sized particles of silica aerogel commercialized under the name Lumira®.

In an embodiment, the at least one non-invasive imaging technique is ultrasound imaging. In such case, the at least one sensing component is selected from gas-containing vesicles as described herein, gas-containing microbubbles as described herein, salt containing particles containing a salt that generates a gas upon contact with a biological fluid and/or an acid and/or hydrogen peroxide, metal or metal oxide nanoparticles, including ZnOx (x between 0 to 2, e.g. 1), Fe₂O₃ and Fe₃O₄ nanoparticles, magnetic carbide nanoparticles as described herein, aerogel particles as described herein, xerogel particles as described herein, and combinations thereof. The at least one sensing component is preferably selected from gascontaining vesicles as described herein, gas-containing capsules as described herein, salt containing particles containing a salt that generates a gas upon contact with a biological fluid and/or an acid and/or hydrogen peroxide as described herein, metal or metal oxide nanoparticles including iron, iron oxide, zinc oxide, zinc ferrite, tantalum, tantalum oxide, bismuth oxide, bismuth ferrite, and gold nanoparticles, and combinations thereof, and more preferably selected from gas-containing vesicles as described herein, gas-containing microbubbles as described herein, carbonate containing particles (CaCO₃, NaHCO₃, and Na₂CO₃), sulphide containing particles (Na₂S, NaHS, and CaS), magnetic carbide nanoparticles, and combinations thereof.

Suitable sensing components that lead to a detectable signal upon application of ultrasound may be selected from the group consisting of gas-containing vesicles and/or gas bubbles of natural or synthetic origin, carbonates such as CaCO₃, NaHCO₃, Na₂CO₃, and other inorganic nanoparticles such as metal or metal oxide, carbide, and/or nitride nanoparticles, in particular ZnOx nanoparticles (x between 0 to 2, e.g. 1).

In some embodiments, carbonates are carbonate nanoparticles. However, carbonates are not limited to carbonate nanoparticles but can vary in size.

Such detectable ultrasound signal can be generated by various means, e.g. the generation, release or collapse of gas containing structures, leading to a detectable change in ultrasound contrast.

The release or generation of gas containing structures is also referred to herein as a “turn-on” type ultrasound signal because the generation or release of said gas containing structure leads to the generation of an ab initio not detectable signal. For example, in case of NaHCO₃ particles, CO₂ is released upon contact with gastric fluid. In a preferred embodiment, NaHCO₃ is used as a sensing component.

Alternatively, the collapse of gas containing structures is also referred to herein as a “turn-off” type ultrasound signal because said collapse leads to the loss of an ab initio detectable signal. For example, gas-containing vesicles and/or gas bubbles that are embedded in the patch (and are ab initio detectable by ultrasound imaging) are collapsed as a result of the contact with biological fluid.

It is to be understood that the terms “turn-on”/“turn-off” do not imply a binary situation but rather indicate a detectable change of a signal.

In a preferred embodiment, gas-containing vesicles and/or gas bubbles are used as a sensing component.

In a particularly preferred embodiment, gas-containing vesicles are used as a sensing component.

In a further preferred embodiment, inorganic nanoparticles, in particular ZnOx (x between 0 to 2, e.g. 1), Fe₂O₃ and/or Fe₃O₄ nanoparticles, are used as a sensing component.

A further non-invasive imaging technique suitable for detecting the leak of the biological fluid at an application site of the patch according to the present invention is computer tomography. In such case, the at least one sensing component is selected from salt containing particles, such as particles containing a radio-opaque salt (e.g.: barium sulphate particles; barium carbonate particles preferably at high concentration) and calcium carbonate particles, preferably at high concentration, metal or metal oxide nanoparticles including iron, iron oxide, zinc oxide, zinc ferrite, tantalum, tantalum oxide, bismuth oxide, bismuth ferrite, and gold nanoparticles, radio-opaque iodinated or brominated organic compounds as described herein, aerogel particles as described herein, xerogel particles as described herein, poly(vinylpyrrolidone)iodine complex particles, and combinations thereof, preferably selected from metal or metal oxide nanoparticles, such as, iron, iron oxide, zinc oxide, zinc ferrite, tantalum, tantalum oxide, bismuth oxide, bismuth ferrite, and gold nanoparticles, barium sulphate particles, poly(vinylpyrrolidone)-iodine complex particles, and mixtures thereof, more preferably selected from barium sulphate particles and tantalum oxide nanoparticles.

Suitable sensing components that lead to a detectable signal upon application of computer tomography may be selected from the group consisting of metal or metal oxide nanoparticles such as iron or iron oxide, zinc oxide zinc ferrite, tantalum or tantalum oxide, bismuth oxide or ferrite, Au nanoparticles or PVP-iodine.

A further non-invasive imaging technique suitable for detecting the leak of the biological fluid at an application site of the patch according to the present invention is magnetic particle imaging (MPI). For such non-invasive imaging technique, the at least one sensing component is selected from magnetic metal or metal oxide nanoparticles, including iron, iron oxide, zinc oxide, zinc ferrite, manganese oxide, terbium oxide, gadolinium oxide, and cobalt ferrite nanoparticles, magnetic carbide nanoparticles, such as iron carbide and iron cobalt carbide nanoparticles, and combinations thereof, preferably iron carbide nanoparticles, such as iron carbide and iron cobalt carbide nanoparticles. The magnetic metal or metal oxide nanoparticles and the magnetic carbide nanoparticles may be surface-modified. Preferably, the magnetic metal or metal oxide nanoparticles, and the magnetic carbide nanoparticles are linked to the synthetic polymer and/or the natural polymer via a hydrolytically cleavable linker and/or an enzyme-digestible peptide.

A further non-invasive imaging technique suitable for detecting the leak of the biological fluid at an application site of the patch according to the present invention is magnetic resonance imaging (MRI). For such non-invasive imaging technique, the at least one sensing component is selected from magnetic metal or metal oxide nanoparticles, including iron, iron oxide, zinc oxide, zinc ferrite, manganese oxide, terbium oxide, gadolinium oxide, and cobalt ferrite nanoparticles, optionally functionalized with a T1 contrast agent, magnetic carbide nanoparticles, such as iron carbide and iron cobalt carbide nanoparticles, optionally functionalized with a T1 contrast agent, gadolinium complexes as described herein, and combinations thereof.

Suitable sensing components that lead to a detectable signal upon application of CT (and/or MRI) may be selected from the group consisting of metal or metal oxide nanoparticles, such as iron or iron oxide, zinc oxide, zinc ferrite, manganese oxide, terbium oxide, gadolinium oxide, surface-modified metal and/or metal oxide nanoparticles, and Gd complexes.

Suitable sensing components that lead to a detectable signal upon application of radio-frequency identification (RFID) may be selected from the group consisting of TWC-401-100 Mifare S50, cellulose acetate, copper wire, sodium carboxymethylcellulose, polyimide or magnesium wire.

The patch claimed and described herein may contain at least one sensing component that is detectable by at least two or more non-invasive imaging techniques. Examples of such sensing components include magnetic carbide nanoparticles. The patch claimed and described herein may contain at least two different sensing components, wherein each of said at least two different sensing components is detectable by a different non-invasive imaging techniques.

The patch claimed and described herein may comprise an additional hydrogel support layer for sealing the application site. The hydrogel support layer comprises a non-degradable synthetic hydrogel comprising one or more monomeric units selected from the group consisting of methacrylates, acrylates, vinyls, thiols, polyurethane forming monomers and mixtures thereof, preferably selected from the group consisting of methacrylates, acrylates, vinyls, and thiols.

The hydrogel support layer may further comprises one or more pre-made synthetic polymers and/or natural polymers and/or acrylated natural polymers. Such additional polymers may be added to fine-tune the properties of the hydrogel support layer such as stability or compatibility with tissue, e.g. by maintaining mechanical stiffness over time.

Suitable methacrylates may be selected from the group consisting of methacrylic acid (MA), methyl methacrylate, methacrylamide, hydroxyethylmethacrylate, ethyl hexyl (meth)acrylate, glycidyl methacrylate, oligoethylene-glycol methylacrylate, 2-(dimethylamino)ethyl methacrylate, [2-(methacryloyloxy)ethyl] trimethylammonium chloride, N-(2-hydroxypropyl) methacrylamide, methacrylic anhydride, N,N-diethylmethacrylamide, (hydroxyphenyl) methacrylamide, 2-hydroxypropyl methacrylamide, 2-aminoethylmethacrylamide hydrochloride, methacryloyl-L-Lysine, phosphoric acid 2-hydroxyethyl acrylate ester, 4-methacryloxyethyl trimellitic anhydride, 3-sulfopropyldimethyl-3-methacrylamidopropylammonium, methacryloyloxyethyl phosphorylcholine (MPC), 2-N-morpholinoethyl methacrylate, 2-aminoethyl methacrylate hydrochloride, methacryloyl-L-lysine, pyridyl disulfide ethyl methacrylate, N-(3-aminopropyl) methacrylamide hydrochloride, N-(3-BOC-aminopropyl) methacrylamide, O-nitrobenzyl methacrylate, O-nitrobenzyl ethyl methacrylate. It is also envisaged to use combinations of two or more methacrylates.

Preferably, methacrylates are selected from the group consisting of methacrylic acid, methyl (methacrylate), methacrylamide, hydroxyethylmethacrylate, ethyl hexyl (meth)acrylate, glycidyl methacrylate, oligoethylene-glycol methylacrylate, 2-(dimethylamino)ethyl methacrylate, [2-(methacryloyloxy)ethyl] trimethylammonium chloride, N-(2-hydroxypropyl) methacrylamide, methacrylic anhydride, N,N-diethylmethacrylamide, 2-hydroxypropyl methacrylamide, methacryloyloxyethyl phosphorylcholine (MPC), 2-aminoethyl methacrylate hydrochloride, pyridyl disulfide ethyl methacrylate, O-nitrobenzyl methacrylate and O-nitrobenzyl ethyl methacrylate and mixtures thereof.

Suitable acrylates may be selected from the group consisting of acrylic acid (AA), acrylamide (AAm), hydroxyethylacrylate, ethyl hexyl acrylate, butyl acrylate, 2-(dimethylamino)ethyl acrylate, (acrylamidopropyl)trimethylammonium chloride, N-(2-hydroxypropyl) acrylamide, N-acryloyl glycinamide, acrylated adenine, acrylated thymine, acrylated cytosine, acrylated guanine, acrylated uracyl, NHS acrylate, NHS sulfo acrylate, acrylic anhydride, 2-acrylamido-2-methyl-1-propanesulfonic, N,N-diethylacrylamide, (hydroxyphenyl) acrylamide, 2-hydroxypropyl acrylamide, Nisopropylacrylamide, beta-carboxyethyl acrylate, 2-N-morpholinoethyl acrylate, 2-aminoethyl acrylate hydrochloride, N-[3-(N,N-dimethylamino) propyl] acrylamide, 2-acryloxyethyltrimethylammonium chloride, 2-cyanoethyl N-acrylate, acryloxysuccinimide, O-nitrobenzyl acrylate, O-nitrobenzyl ethyl acrylate. It is also envisaged to use combinations of two or more acrylates.

Preferably, acrylates are selected from the group consisting of acrylic acid, acrylamide, hydroxyethylacrylate, ethyl hexyl acrylate, butyl acrylate, 2-(dimethylamino)ethyl acrylate, (acrylamidopropyl)trimethylammonium chloride, N-(2-hydroxypropyl) acrylamide, N-acryloyl glycinamide, NHS acrylate, NHS sulfo acrylate, acrylic anhydride, N,N-diethylacrylamide, (hydroxyphenyl) acrylamide, 2-hydroxypropyl acrylamide, N-isopropylacrylamide, beta-carboxyethyl acrylate, 2-aminoethyl acrylate hydrochloride, N-[3-(N,N-dimethylamino) propyl] acrylamide, 2-acryloxyethyltrimethylammonium chloride, 2-cyanoethyl acrylate, Nacryloxysuccinimide, O-nitrobenzyl acrylate, and O-nitrobenzyl ethyl acrylate, and mixtures thereof.

Suitable vinyls may be selected from the group consisting of vinyl pyrrolidinone, vinylcaprolactam, sodium styrene sulfonate, 2-methylene-1,3-dioxepane, 3-(acrylamido)phenylboronic acid, allyl methyl sulfone, 3-allyloxy-2-hydroxy-1-propanesulfonic acid sodium salt, 3-allyloxy-1,2-propanediol, N-methyl-N-vinyl acetamide, N-vinyl acetamide (NVA), vinylphosphonic acid, allylphosphonic acid monoammonium salt, mutliarm PEG (2 or 3 or 4 arms) vinyl sulfone. It is also envisaged to use combinations of two or more vinyls.

Preferably, vinyls are selected from the group consisting of vinyl pyrrolidinone, vinylcaprolactam, and sodium styrene sulfonate, and mixtures thereof.

Suitable thiols may be selected from the group consisting of 1,4-butanedithiol, 1,3-propanedithiol, 1,2-ethanedithiol, 2,2′ (ethylenedioxy) diethanethiol, ethylene glycol bis-mercaptoacetate, 2-arm PEG-SH, 3-arm PEG-SH, 4-arm PEG-SH. It is also envisaged to use combinations of two or more thiols.

Preferably, thiols are selected from 1,3-propanedithiol and 2,2′ (ethylenedioxy) diethanethiol, and mixtures thereof.

Polyurethanes are formed by reactions of polyols and isocyanates. Suitable urethane forming monomers may be selected from the lists below. Again, two or more polyols and two or more isocyanates may be used to form the hydrogel.

Polyols: 2,4,6-Tria-mino-1,3,5-triazine, 3,5-dimethoxy-4-hydroxybenzaldehyde, polyethylene glycol (PEG 400, 600, 1000), 1,4-butanediol, CAPA™3050.

Isocyanates: Hexamethylene diisocyanate, sophoronediisocyanat, L-lysine ethyl ester diisocyanate, isophorone diisocyanate.

Suitable natural polymers may be selected from the group consisting of alginate, chitosan, carboxymethylcellulose, dextran, heparin, hyaluronic acid, gellan gum, and kappa-carraggennans.

Suitable acrylated natural polymers may be selected from the group consisting of acrylated elastin, acrylated albumin, acrylated alginate and acrylated human serum albumin.

Suitable pre-made synthetic polymers may be selected from the group consisting of polyacrylic acid, poly(N-(2-hydroxypropyl) methacrylamide) (PHPMA), polyvinylacetate (PVA), polycaprolactone (PCL), poly(2-hydroxyethyl methacrylate) (PHEMA), polyvinylidene difluoride (PVDF), polyethylene glycol (PEG 400, 600, 1000), polydopamine, polygallic acid, hydroxyethylmethacrylate.

Preferably, pre-made synthetic polymers are selected from the group consisting of polyacrylic acid, PHPMA, PVA, PHEMA, PVDF, polyethylene glycol (PEG 400, 600, 1000), polydopamine, and polygallic acid.

Typically, one or more monomeric units comprised within said non-degradable, synthetic hydrogel function as crosslinkers. Such crosslinkers are known in the field and selected in accordance with the monomer units of the hydrogel.

Some monomers, particularly monomers that have relatively strong H-bond interactions such as N-acryloyl glycinamide (NAGA) do not require the addition of crosslinkers to form a hydrogel. However, most monomers require addition of crosslinkers to form a hydrogel.

As convenient in the field, polymers may comprise different classes of monomers as mentioned above, e.g. copolymers of acrylates and methacrylates.

Suitable crosslinkers may be selected from the group consisting of polyethylene glycol dimethacrylate (particularly PEGDMA 20000), 1,6-hexanediol diacrylate, 1,4-butanediol diacrylate, 1,4-phenylene diacrylate, bis(2-methacryloxyethyl) phosphate, triethylene glycol diacrylate (TriEGDA), dipentaerythritol pentaacrylate, 1,1,1-trimethylolpropane triacrylate (TriMPTA), 1,1,1-trimethylolpropane trimethacrylate, PEO (5800)-b-PPO (3000)-b-PEO (5800) dimethacrylate, pentaerythritol triacrylate, pentaerythritol tetraacrylate, tricyclodecane dimethanol diacrylate, PEGDA-PEG diacrylate, zinc dimethacrylate, carboxybetaine disulfide cross-linker (CBX-SS), oxidized alginate, diselenide crosslinker, 2,2-dimethacroyloxy-1-ethoxypropane (DMAEP), N,N′-bis(acryloyl) cystamine (BAC), methylene bis-acrylamide (mBAA), 1,3,5-triallyl-1,3,5-triazine-2,4,6 (1H,3H,5H)-trione, mutliarm PEG (2,3,4) thiol terminated, multiarm PEG (2 or 3 or 4 arms) vinyl sulfone terminated, multiarm PEG (2,3,4) NHS terminated, multiarm PEG (2 or 3 or 4 arms) NH2 terminated, 2,4,6-triamino-1,3,5-triazine, 3,5-dimethoxy-4-hydroxybenzaldehyde, polyethylene glycol (PEG 400, 600, 1000).

Preferably, crosslinkers are selected from the group consisting of polyethylene glycol dimethacrylate (PEGDMA 20000), 1,4-butanediol diacrylate, pentaerythritol triacrylate, PEGDA-PEG diacrylate, zinc dimethacrylate, carboxybetaine disulfide cross-linker (CBX-SS), oxidized alginate, 2,2-dimethacroyloxy-1-ethoxypropane (DMAEP), N,N′-Bis(acryloyl) cystamine (BAC), methylene bis-acrylamide, multiarm PEG (2 or 3 or 4 arms) vinyl sulfone terminated, and multiarm PEG (2 or 3 or 4 arms) NHS terminated, and mixtures thereof.

Preferably the hydrogel support layer comprises a non-degradable, synthetic hydrogel comprising monomeric units selected from the group consisting of acrylamide, acrylic acid, methyl acrylate, 2-hydroxyethyl acrylate, 2-hydroxyethyl acrylamide, sodium 2-acrylamido-2-methylpropane sulfonate, N-acryloyl glycinamide, styrene sulfonate, N-Tris(hydroxymethyl)methyl acrylamide, bis-acrylamide, poly ethylene glycol diacrylate, N,N′-bis(acryloyl) cystamine, and mixtures thereof.

The hydrogel support layer may comprise a non-degradable, synthetic poly(AAm-AA-MA) hydrogel (p (AAm-AA-MA)), comprising AAm and AA and MA and mBAA monomeric units, wherein mBAA functions as a crosslinker.

The patch claimed and described herein may comprise an additional backing layer. Preferably, the patch claimed and described herein consists of the layer described at i), or ii) or the layers described at iii) and the backing layer described herein. Backing layers are well known in the art. They are included to provide structural integrity and/or additional functionality as described below. The backing layer may comprise a non-adhesive polymer. Suitable backing layers can generally be prepared from the same materials as described for the hydrogel support layer, including natural polymers and/or acrylated natural polymers as described for hydrogel support layers. However, the specific composition of the backing layer differs from the hydrogel support layer.

The skilled person is capable of selecting a backing layer comprising a nonadhesive polymer using the above materials, including the monomeric units in a suitable ratio and/or natural polymers and/or acrylated natural polymers.

Said non-adhesive polymer can be a non-adhesive synthetic polymer comprising monomeric units selected from the group consisting of methacrylates, acrylates, vinyls, thiols, polyurethane forming monomers, and mixtures thereof in a suitable ratio and/or a suitable natural polymer and/or acrylated natural polymer. In particular, non-adhesive properties can be conferred on the backing layer by including carboxymethylcellulose (CMC), poly(N-acryloyl glycinamide) (PNAGA), poly(hydroxyethyl acrylate) (PHEA), poly(N-(2-hydroxyethyl) acrylamide) (PNHEAA).

Preferably, monomeric units comprised within said non-adhesive synthetic polymer are selected from the group consisting of methacrylates, acrylates, vinyls, thiols, and mixtures thereof.

Preferably the non-adhesive polymer is selected from PNHEA (poly(Nhydroxyethylacrylamide)), and polyurethane, including hydrophilic polyurethanes, such as HydroMed D4 polyurethane.

Typically, the backing layer comprises 2%-90% water. Preferably, the backing layer comprises 5%-80% water, more preferably, 5%-60% water, such as 5%, 10%, 15%, 20%, 25%, 30%, 35%, 40%, 45%, 50%, 55% or 60% water.

The patch claimed herein may additionally comprise one or more therapeutically active components. Said therapeutic components may be suitable for treating a leak at an anastomosis site, e.g. therapeutic components with antibiotic or wound healing activity. Preferably, said therapeutic components that are suitable for treating a leak at an anastomosis site, e.g. antimicrobials, are released from the patch upon contact with intestinal fluid, e.g. through swelling and/or (full or partial) degradation of the hydrogel matrix.

Suitable therapeutic components with antimicrobial activity may be selected from the group consisting of antimicrobials including beta-lactams (penicillins), carbapenems (beta-lactamase-resistant beta-lactams), cephalosporins (semi-synthetic beta-lactams), and quinolones (e.g. ciprofloxacin), glycopeptides (e.g. vancomycin), aminoglycosides (e.g. gentamycin), and mupirocin and mixtures thereof in free or immobilized form, including biopolymer grafted antimicrobials, such as alginate grafted gentamycin. Further therapeutic components include metal and/or metal oxide nanoparticles or salts, particularly Ag/AgOx, ZnOx, Cu/CuOx, GaOx, cerium chloride, supported metal and metal oxide nanoparticles, including SiO2/Ag, SiO2/Cu, SiO2/ZnOx, functionalized metal and/or metal oxide nanoparticles, and mixtures thereof, wherein x can be between 0 to 2, e.g. 1.

Preferably, said therapeutic components are selected from the group consisting of Ag, ZnOx (x between 0 to 2, e.g. 1).

For example, such therapeutic components are suitable for the treatment of septic peritonitis that may result from leaking of bacteria-containing intestinal fluid.

In a further preferred embodiment, said therapeutic components are suitable to support wound healing at a sutured or stapled site, particularly an anastomosis site.

Therapeutic components that are suitable to support wound healing at a sutured or stapled site, particularly an anastomosis site may be selected from the group consisting of growth factors such as platelet-derived growth factor (PDGF), transforming growth factor-β1 (TGF-β1), vascular endothelial growth factor (VEGF), fibroblast growth factor (FGF), growth hormone (GH), flavonoids, metal oxide nanoparticles and salts, alginate, and peptides such as RGD, TB 4, AP-57, and mixtures thereof. The above compounds are integrated into the hydrogel either by passive loading, crosslinking, conjugation to a natural or synthetic polymer backbone or by incorporating drug loaded liposomes or polymersomes.

However, in light of the above disclosure, it is clear to the skilled person that various other therapeutic components can be used in the context of the present invention.

The skilled person, taking into consideration the common technical knowledge in the medical field, would know and/or select the additional therapeutic component in light of the disease/condition to be treated.

Said therapeutic components may be present in any layer of the patch as described above.

Said therapeutic components may be encapsulated in an additional matrix, which is referred to as an encasing therapeutic matrix. The therapeutic components may be finely dispersed within at least one layer of the patch.

The encasing therapeutic matrix may comprise a synthetic polymer having monomeric units selected from the group consisting of 2-acryloyloxy)ethyl)trimethylammonium chloride), acrylamide, acrylic acid, methyl acrylate, 2-hydroxyethyl acrylate, 2-hydroxyethyl acrylamide, sodium 2-acrylamido-2-methylpropane sulfonate, N-acryloyl glycinamide, styrene sulfonate, Ntris(hydroxymethyl) methyl acrylamide, bis-acrylamide, poly ethylene glycol diacrylate, N,N′-bis(acryloyl) cystamine, and mixtures thereof.

The encasing therapeutic matrix may comprise one or more natural polymers selected from the group consisting of carboxymethylcellulose, alginate, agar, bovine serum albumin, human serum albumin, gelatine.

The release of therapeutic components may occur due to swelling of the polymer matrix or cleavage of the therapeutic component from its polymer matrix (full or partial degradation of said polymer matrix) located within the hydrogel support layer and/or the backing layer and/or the encasing therapeutic matrix.

A second aspect according to the present invention is directed to a patch for detecting by at least one non-invasive imaging technique a leak of a biological fluid at an application site, said patch comprising:
a first layer containing a first gel;
a second layer containing a second gel; and
wherein at least one of the first layer and the second layer, preferably both the first and the second layer, contains a sensing component embedded within the gel contained therein;
the at least one sensing component being selected from gas-containing vesicles as described herein, gas-containing microbubbles as described herein, salt containing particles as described herein, metal or metal oxide nanoparticles as described herein, carbide nanoparticles as described herein, radio-opaque iodinated or brominated organic compounds as described herein, gadolinium complexes as described herein, monosaccharide containing microspheres as described herein, polysaccharide containing microspheres as described herein, protein containing microspheres as described herein, poly(vinylpyrrolidone)-iodine complex particles as described herein, and combinations thereof;
the first gel and the second gel being selected from the group consisting of a hydrogel, an aerogel, and a xerogel, and comprising a synthetic polymer as described herein, a natural polymer as described herein, or a mixture thereof; and
wherein
iii-1) the first gel is non-degradable and the second gel is degradable, or
iii-2) the first gel and the second gel are non-degradable, the first gel has a relative swelling ratio, and the second gel has a relative swelling ratio, and the relative swelling ratio of the first gel is different from the relative swelling ratio of the second gel, with the relative swelling ratio being the ratio between the mass of said gel after incubation for 24 h at 37° C. in a simulated intestinal fluid and the mass of said gel prior to incubation in the simulated intestinal fluid.

The first layer and the second layer described herein are preferably overlapping.

The first gel and the second gel are preferably hydrogels.

In embodiment iii-1), the non-degradable first gel comprises preferably one or more of the synthetic polymers described herein and/or one or more of the nondegradable natural polymers described herein, such as cellulose or carboxymethylcellulose, more preferably one or more of the synthetic polymers described herein having monomeric units selected from the group consisting of acrylamide, acrylic acid, methyl acrylate, 2-hydroxyethyl acrylate, 2-hydroxyethyl acrylamide, sodium 2-acrylamido-2-methylpropane sulfonate, N-acryloyl glycinamide, styrene sulfonate, Ntris(hydroxymethyl) methyl acrylamide, bis-acrylamide, polyethylene glycol diacrylate, N,N′-bis(acryloyl) cystamine, and mixtures thereof, most preferably one or more synthetic polymers selected from polyacrylamide, poly(2-acrylamido-2-methyl-1-propanesulfonic acid), poly(N-(2-hydroxyethyl) acrylamide) (PNHEA), poly(acrylamide-co-methyl acrylate-co-acrylic acid), and poly(methylacrylate-coacrylic acid. The degradable second gel comprises preferably a degradable natural polymer, such as bovine serum albumin, human serum albumin, agar, or alginate, cholic acid-alginate, or a degradable synthetic polymer having a crosslinked network, such as PNHEA-BAC.

In embodiment iii-2), preferably, there is a difference of at least 3 in absolute value between the relative swelling ratio of the first gel and the relative swelling ratio of the second gel. More preferably, there is a difference of at least 4, such as at least 5, at least 10, at least 15, or at least 20, in absolute value between the relative swelling ratio of the first gel and the relative swelling ratio of the second gel. The nondegradable first gel and the non-degradable second gel comprise preferably one or more of the synthetic polymers described herein and/or one or more of the nondegradable natural polymers described herein, such as cellulose or carboxymethylcellulose, more preferably one or more of the synthetic polymers described herein having monomeric units selected from the group consisting of acrylamide, acrylic acid, methyl acrylate, 2-hydroxyethyl acrylate, 2-hydroxyethyl acrylamide, sodium 2-acrylamido-2-methylpropane sulfonate, N-acryloyl glycinamide, styrene sulfonate, Ntris(hydroxymethyl) methyl acrylamide, bis-acrylamide, polyethylene glycol diacrylate, N,N′-bis(acryloyl) cystamine, and mixtures thereof. Most preferably, the nondegradable first gel comprises one or more synthetic polymers selected from PAMPS, PHNEA, P (AA-MA), P (AAm-MA-AA), and PAAm. Most preferably, the nondegradable second gel comprises one or more of the synthetic polymers selected from PNAGA, PAAm, Hydromed D1, Hydromed D2, Hydromed D3, Hydromed D4, Hydromed D6, Hydromed D640, Hydromed D7 and HydroSlip C. The nondegradable first gel and the non-degradable second gel are to be chosen so that their relative swelling ratios are different. A difference of at least 3, such as of at least 4, 5, 10, 15, or 20, in absolute value between the relative swelling ratio of the first gel and the relative swelling ratio of the second gel is preferred.

A third aspect according to the present invention relates to a method of detecting by at least one non-invasive imaging technique a leak of a biological fluid at an application site in a subject in need thereof.

Preferably, the application site is selected from a suture site, a stapled site or an implantation site of a surgical implant.

Preferably the application site is located in the abdominal region. More preferably, the application site is selected from an artificial stoma site, such as a permanent stoma site or a temporary stoma site, an intestinal anastomosis site, a stomach resection site, a gallbladder anastomosis site, a gallbladder resection site, a liver resection site, a colon resection site, a colon anastomosis site, a pancreas resection site, a pancreas anastomosis site and a portacaval anastomosis site.

Preferably, the non-invasive imaging technique is selected from at least one of ultrasound imaging, computer tomography, magnetic resonance imaging and magnetic particle imaging.

In one embodiment, the method of detecting by at least one non-invasive imaging technique a leak of a biological fluid at an application site comprises the following steps in the order as indicated:
a) Applying the patch as described herein to the application site;
b) Measuring a signal by a non-invasive imaging technique, preferably selected from at least one of ultrasound imaging, computer tomography, magnetic resonance imaging, magnetic particle imaging and radio-frequency identification at a determined time point; and
c) Detecting a contrast between the i) one or more first portions and the one or more second portions, or ii) the third gel and the aerogel particles, or xerogel particles embedded by said third gel, or iii) the first layer and the second layer. A determined time point may be any suitable time point following the application of the patch claimed and described herein to the application site. The determined time point may be for example in between 1 hour and several days, more preferably in between 1 hour and 24 hours following application of the patch to the application site. Steps b) and c) may be repeated.

In another embodiment, the method of detecting by at least one non-invasive imaging technique a leak of a biological fluid at an application site comprises the following steps in the order as indicated:
a) Applying the patch as described herein to the application site;
b) Measuring a signal by a non-invasive imaging technique, preferably selected from at least one of ultrasound imaging, computer tomography, magnetic resonance imaging and magnetic particle imaging at a first time point;
c) Measuring a signal by a non-invasive imaging technique, preferably selected from at least one of ultrasound imaging, computer tomography, magnetic resonance imaging and magnetic particle imaging at a second time point;
d) Comparing the signal measured at a first time point with the signal measured at a second time point. A first time point may be any suitable time point following the application of the patch claimed and described herein to the application site. The first time point may be immediately following application of the patch to the application site or may be 24 hours following application of the patch to the application site. The second time point may be any suitable time point following the first time point, such as 1 hour, 2 hours, 24 hours, or several days after the first time point. Steps b)-d) may be repeated.

To further illustrate the invention, the following examples are provided. These examples are provided with no intend to limit the scope of the invention.

All materials used for the examples outlined below were purchased from SigmaAldrich (Merck). Acrylic acid (AA) and methyl acrylate (MA), N-hydroxyethyl acrylamide (NHEA), monomers were purified by passing them through a plug of basic alumina (Brockmann Grade I). Acrylamide (AAm) and N,N′-methylenebisacrylamide (mBAA) were used without further purification. The specific dimensions of the teflon molds that were used for shaping various layers of the patch are selected based on the intended application and experimental setup. Fresh small intestines were cleaned of its contents manually, divided into pieces and then stored at −20° C. Intestines were preferably used fresh or thawed once and subsequently used for experiments.

### 1. Preparation of a P (AAm-MA-AA) Hydrogel as a Non-Degradable Hydrogel Support Layer for the Patch Claimed Herein

First, stock solutions of monomer mixes were prepared and then used to assemble master mixes.

1.a A stock solution of acrylamide (AAm) monomer was made by dissolving the powder in milliQ water at 20 wt %. A 2 wt % crosslinker was made by dissolution of 0.2 g (1.3 mmol) N,N′-methylenebisacrylamide (mBAA) dissolved in 9.8 g Milli-Q water. Both stock solutions were kept for a maximum of 30 days stored at 0-4° C. Stock solutions of curing initiators (photoinitiators) were made fresh before each experiment and kept in the dark. 9.5 mg (42 μmol) 2-hydroxy-4′-(2-hydroxyethoxy)-2-methylpropiophenone (Irgacure 2959) were dissolved in 2 mL Milli-Q water by sonication for 20 min. Previously cleaned monomers were then used to assemble the final hydrogel master mix.

6 mL of AAm monomer stock solution was mixed with 2.28 mL of AA, 1.25 mL of MA, 0.6 mL of photoinitiator Irgacure D-2959 solution and 61.7 μL of mBAA crosslinker stock solution. All constituents were then vortexed in a 15 mL falcon tube. From the resulting mix 300 μL were spread to a Teflon round mold (diameter: 20 mm, depth: 1 mm). The mold was then put under a UVASPOT 400/T mercury lamp at a distance of 30 cm from the source. The light source was equipped with a filter (H2) that allows the spectrum interval from 300 nm until the visible light range to reach the hydrogel mix. The P (AAm-MA-AA) hydrogel was obtained after 180 s irradiation. The resulting hydrogel was then either used directly for further layering or dialyzed against MilliQ water or PBS 4× over 24 h.

1b. Preparation of a Hydrogel Support Layer Comprising a Non-Degradable PAMPS Hydrogel

An AMPS monomer mix consisting of 4 mL of 50 wt % of AMPS (ali-quoted from the as received stock), 20 μL of mBAA stock and 30.6 μL of Irgacure stock solution, was prepared and used to add 300 UL of this latter to the circular Teflon mold (2 cm diameter, 0.2 cm thickness). After allowing the solution to settle for 1 min, the novel layer was polymerized as previously described for 5 min

### 2. Preparation of a Non-Adhesive Polyacrylamide Backing Layer for the Patch Claimed Herein

In case the patch comprises a backing layer, at least two different methods of preparing said backing layer have been used.

2a. Preparation of a Non-Adhesive Polyacrylamide Backing Layer Directly on a Patch Layer as Described Herein

A 20 wt % acrylamide stock solution of 5 mL was mixed with 108 μL of 2 wt % mBAA crosslinker solution and 500 μL of photoinitiator Irgacure 2959 (4.825 mg/mL). The resulting prepolymer solution was then cast directly on the top face of the hydrogel support layer described by example 1 and cured under a UVASPOT 400/T mercury lamp as de-scribed above.

2b. Preparation of a Separate Non-Adhesive Polyacrylamide Backing Layer for Assembly with a Patch Layer as Described Herein

A 20 wt % acrylamide stock solution of 5 mL was mixed with 108 μL of 2% mBAA crosslinker solution and 500 μL of photoinitiator Irgacure 2959 (4.825 mg/mL). The resulting prepolymer solution was then cured independently from the hydrogel support layer under a UVASPOT 400/T mercury lamp as described above. The resulting backing layer was assembled with the hydrogel support layer at a later stage by adding a fresh mix of the above described prepolymer solution as described above at the inter-face between the hydrogel support layer and the backing layer.

2c. Preparation of a Non-Adhesive PNHEA Backing

A backing layer comprising a non-adhesive PNHEA polymer was prepared in analogy to examples 2a and 2b. 300 μL of a polymerizable stock solution composed of pure, inhibitor removed, 2 mL NHEA monomer, mixed with 2 mL of milliQ water, 53.32 UL of mBAA 2 wt % stock solution and 302 μL of the Irgacure solution (example 1a), the monomer mix was spread on the underlying support layer and left to diffuse in the support layer for 1 min before a 5 min polymerization step. The prepared patches were then kept in the mold and protected from drying using a polyethylene foil until application.

### 3. Preparation of Gels Comprising Gas-Containing Vesicles as Sensing Components

3a. Preparation of Polyacrylamide (PAAm) Gel Comprising Gas-Containing Vesicles as Sensing Components Embedded Therein for Generating a “Turn-Off” Type Signal by Ultrasound for the First Portions of the Patch Claimed Herein

Gas-containing vesicles were presented as milky suspensions in PBS buffer with a typical optical density (OD) of 18. This suspension was then used to dilute acrylamide monomers at 20% vol. A hydrogel comprising 20% vol gas-containing vesicle as sensing components was made by diluting 1 mL of PBS gas-containing vesicle suspension with 4 mL of MilliQ water, adding to the obtained suspension 1 g of acrylamide, 54 μL of mBAA 2% solution and 8 μL TEMED polymerization accelerator, placing 150 μL of the resulting mix in a Teflon mold and polymerizing by adding 5.8 μL of a 60 mg/mL ammonium persulfate MilliQ water solution. The resulting mixture was placed at 60° C. for 4 min. The resulting opaque white hydrogel was lifted with Teflon tweezers, covered with para-film to avoid drying and stored at 2-8° C. for further use. The embedded gas-containing vesicles (FIG. 3b (ii)) are digested upon contact with intestinal fluid (“turn-off” type signal; FIG. 3b).

3b. Preparation of a Polyacrylamide (PAAm) Gel Having Gas-Containing Vesicles as Sensing Components and Silica Powder as Contrast Agent Embedded Therein for Generating a “Turn-Off” Type Signal with Negative Contrast.

In a first step a precursor composition for a hydrogel comprising 33% vol gascontaining vesicles as sensing components was prepared in a similar fashion as described in example 3a. The PBS gas-containing vesicle suspension was diluted with 4 mL of MilliQ water to which is added 1 g of acrylamide, 54 L of mBAA 2% solution and 375 μL of photoinitiator LAP (6.55 mg/mL).

In a second step precursor composition for a hydrogel comprising 2% silica powder was prepared. Herefore 80 mg of silica powder were suspended in 4 mL of MilliQ water. To that is added 1 g of acrylamide, 54 μL of mBAA 2% solution and 375 L of photoinitiator LAP (6.55 mg/mL).

In a third step pure acrylamide hydrogel precursor composition was prepared by adding 1 g of acrylamide, 54 μL of mBAA 2% solution and 375 μL of photoinitiator LAP (6.55 mg/mL) to 4 mL of MilliQ water.

The complete layer was assembled by sequentially forming the three individual gels on top of each other inside a cylindrical Teflon mold of 300 μL volume and placing the mold under a UV lamp (2×6W-365 nm VL-206.BL lamp). The bottom part of the layer consisted of 40 μL of the gas-containing vesicles hydrogel precursor composition. The middle part of the layer consisted of 80 μL of pure acrylamide hydrogel precursor composition and the top part of the layer consisted of 40 μL of the hydrogel precursor composition containing the silica powder. The resulting hydrogel was lifted with Teflon tweezers, covered with parafilm to avoid drying and stored at 2-8° C. for further use. The embedded gas-containing vesicles are digested upon contact with intestinal fluid (“turn-off” type signal; FIG. 10b).

3c. Preparation of a Polyacrylamide Gel Having Gas-Containing Vesicles as Sensing Components and Silica Nanoparticles as a Contrast Agent for Generating a “Turn-Off” Type Signal with Negative Contrast.

The precursor composition of a 9% silica containing hydrogel was prepared by adding 7.2 g silica powder into 80 mL of milliQ water along with 20 g acrylamide, 1080 μL mBAA 2% solution and 160 μL TEMED polymerization accelerator.

In addition a precursor composition of a hydrogel comprising 33% vol. gascontaining vesicles as sensing components with photoinitiator LAP was prepared as described above.

100 μL of the precursor composition containing the gas-containing vesicles were formed in a cylindrical Teflon mold under a UV lamp (2×6W-365 nm VL-206.BL lamp). Using Teflon tweezers, the resulting opaque white hydrogel was removed from the mold and placed into the center of a larger cylindrical mold of 6 mL volume. 5.5 mL of the precursor composition containing the silica were carefully added to the mold, making sure not to cover the hydrogel containing the gas-containing vesicles. Polymerization was started by adding 205 μL of a 60 mg/mL ammonium persulfate MilliQ water solution. The mold was placed at 60° C. for 5 min. This results in a sensing matrix semi-encapsulated in a reference matrix. The complete hydrogel was covered in parafilm to avoid drying and stored at 2-8° C. for further use. The embedded gas-containing vesicles are digested upon contact with intestinal fluid (“turn-off” type signal; FIG. 10a).

### 4. Preparation of Gels Comprising Sodium Bicarbonate Particles as Sensing Components

4a. Patch According to the Present Invention Having Polyacrylamide Hydrogel as a First Gel, Agar as a Second Gel, and Sodium Bicarbonate Sensing Component Embedded within the Second Gel

A 2 wt % agar water solution was brought to boil and agar was dissolved. To the hot transparent solution was added 2.5 wt % of sodium bicarbonate powder and the mixture was stirred until homogenous under a hot plate. The resulting warm mixture was then cast on round Teflon molds at 50-300 μL increments and left to cool down. At room temperature the resulting gel was used further for incorporation into a 20 wt % AAm imaging phantom. Sodium bicarbonate leads to the generation of gas bubbles upon contact with gastric fluid (“turn-on” type signal; FIG. 5c and FIG. 12) which are detected using clinical ultrasound.

4b. Patch According to the Present Invention Having a Polyacrylamide Hydrogel as a First Gel, Agar as a Second Gel, and Sodium Bicarbonate Particles as Sensing Component Embedded within the Second Gel

In a hot solution consisting of 2 wt % agar are dissolved under hot conditions (90-100° C.) and under vigorous stirring sodium bicarbonate 2 wt % over the final mass. Upon cooling the formed hydrogel is cut into circular disks using an 8 mm biopsy punch. The formed gels were incubated in PBS to adjust the pH. The resulting gel disks (second gel containing sensing component, 8 mm radius, 2 mm thickness) was then encapsulated in a polyacrylamide gel in the following way: The gel disk was placed into a 2 cm circular Teflon mold. The Teflon mold was then filled with 300 μL of a polymerizable mixture made of 5 mL of 20 wt % Acrylamide, 500 μL 4.825 mg/mL Irgacure D-2959, 64.8 μL mBAA 2 wt % (all solutions were in MilliQ water). The mold was then put under a UVASPOT 400/T mercury lamp at a distance of 30 cm from the source for 5 minutes. The light source was equipped with a filter (H2) that allows the spectrum interval from 300 nm until the visible light range to reach the polymerizable mixture. As a result, the agar gel containing sodium bicarbonate (sensing component embedded in the second gel) was partially encapsulated in the polyacrylamide gel (first gel).

Sodium bicarbonate trapped in agar and partially embedded into polyacrylamide leads to the generation of gas bubbles upon contact with gastric fluid which remain partially trapped within the polyacrylamide matrix (“turn-on” type signal; FIG. 13).

4c. Patch According to the Present Invention Having Polyacrylamide Hydrogel as a First Gel, Agar as a Second Gel, and Sodium Bicarbonate Particles as Sensing Component Embedded within the Second Gel

In a hot solution consisting of 2 wt % agar are dissolved under hot conditions (90-100° C.) and under vigorous stirring sodium bicarbonate particles 2 wt % over the final mass. Upon cooling the formed hydrogel is cut into circular disks using an 8 mm biopsy punch. The formed gels were incubated in PBS to adjust the pH. The resulting gel disks (second gel containing sensing component, 8 mm radius, 2 mm thickness) was then encapsulated in a polyacrylamide gel (second gel-high swelling matrix) in the following way: The gel disk was placed into a 2 cm circular Teflon mold. The Teflon mold was then filled with 300 μL of a polymerizable mixture made of 5 mL of 20 wt % Acrylamide, 500 μL 4.825 mg/mL Irgacure D-2959, 64.8 μL mBAA 2 wt % (all solutions were in MilliQ water). The mold was then put under a UVASPOT 400/T mercury lamp at a distance of 30 cm from the source for 5 minutes. The light source was equipped with a filter (H2) that allows the spectrum interval from 300 nm until the visible light range to reach the polymerizable mixture. As a result, the agar gel containing sodium bicarbonate (sensing component embedded in the second gel) was partially encapsulated in the polyacrylamide gel (first gel). The resulting partiallyencapsulated agar disk was then flipped and an additional 300 μL quantity of the polymerizable mixture described above was added. Polymerization was performed as described above.

Sodium bicarbonate particles trapped in agar and fully embedded into polyacrylamide leads to the generation of gas bubbles upon contact with gastric fluid which remain trapped within the polyacrylamide matrix for long periods of time giving off a discernible signal (“turn-on” type signal; FIG. 14)

4d. Preparation of a Second Gel Comprising NaHCO₃35 wt %-CT as Sensing Component Embedded within Agar for the Second Portions of the Patch Claimed Herein

In a hot solution consisting of 2 wt % agar (second gel-low swelling gel) are dissolved under hot conditions (90-100° C.) and under vigorous stirring sodium bicarbonate particles 35 wt % over the final mass. Upon cooling the formed hydrogel is cut into circular disks using an 8 mm biopsy punch.

Sodium bicarbonate particles at 35 wt % gives a discernable signal under CT prior to contact with simulated gastric fluid, which decreases in intensity following contact with SGF. An additional signal after contact with simulated gastric fluid becomes apparent under ultrasound due to CO₂ formation and entrapment as shown by FIG. 17a).

4e. Preparation of a Second Gel Comprising CaCO₃ Particles 35 wt %-CT as Sensing Component Embedded within Agar for the Second Portions of the Patch Claimed Herein

In a hot solution consisting of 2 wt % agar (second gel-low swelling hydrogel) are dissolved under hot conditions (90-100° C.) and under vigorous stirring calcium carbonate 35 wt % over the final mass. Upon cooling the formed hydrogel is cut into circular disks using an 8 mm biopsy punch.

Calcium carbonate at 35 wt % gives a discernable signal under CT prior to contact with simulated gastric fluid, which decreases in intensity following contact with SGF. An additional signal after contact with simulated gastric fluid becomes apparent under ultrasound due to CO₂ formation and entrapment as shown by FIG. 17b).

4f. Preparation of a Patch Containing PNHEA as a First Gel, PAAm—as a Second Gel and Particles Containing Citric Acid and Sodium Bicarbonate Coated with Beeswax as a Sensing Component Embedded within the Second Gel

Crystalline citric acid and sodium bicarbonate are powdered using a mortar at a 1:1 mass ratio. The resulting powder is then mixed with beeswax pellets at 5 fold mass ratio excess and is then heated at 70° C. under vigorous stirring. The resulting mixture is cooled down while under stirring and the resulting composite material is then once again powdered using liquid nitrogen and a mortar. 0.1 g of the fine powder resulting is then mixed and vortexed with a prepolymer mix consisting of 5 mL of 20 wt % acrylamide, 500 μL 6.33 mg/mL LAP, 64.8 μL mBAA 2 wt % (all solutions are in MilliQ water) and placed as prepared and at 100 μL increments onto a 2 cm circular Teflon. The mix was then polymerized for 5 min under UV lamp (2×6W-365 nm VL-206.BL lamp) to provide a hydrogel with low relative swelling ratio. The formed composite polyacrylamide hydrogels are then left within the same mold and enveloped with 300 μL of a liquid polymerizable mix made of 2 mL NHEA, 2 mL MilliQ water, 53.32 μL mBAA, 301.3 L 4.825 mg/mL 12959 (i.e. the precursor composition for a hydrogel with high relative swelling ratio).

The resulting patch allows the formation of carbon dioxide bubbles upon digestion of the beeswax additive by gastric or intestinal fluids.

4g. Preparation of a Patch Containing PAMPS as a First Gel and Agar Containing 2 wt % NaHCO₃ as a Second Gel and a PNHEA Backing

Sodium bicarbonate sensing elements were prepared in 2 wt % agar in water solution as described in 4a. After homogenization the solution was allowed to cool to room temperature and once gelled was cut in cylinders of 8 mm in diameter using an 8 mm sterile biopsy punch. The cylindrical gels were cut into disks of 0.2 cm thickness using sterile scalpels. The prepared disks were then mounted in an 8 mm hole created at the level of a pre-polymerized PAMPS hydrogel layer to accommodate the sensing disk. The construct was fused together using 300 μL of the polymerizable NHEA backing mix as earlier described. Results can be seen in FIG. 11.

5. Preparation of Patches According to the Present Invention Containing Aerogel Particles as Sensing Components Embedded within a Hydrogel

### 5a.1 Preparation of Alginate Cholate (Cholic Acid-Alginate) Aerogel Particles

A solution of 2M sodium alginate in MilliQ water is created (based on the monomeric units) and mixed with the molar monomeric equivalent quantity to the alginate subunits with sodium cholate. The resulting mix is then divided into 50 μL volumes and to each of those is added a 2 μL of CaCl2 solution of 0.15 M. The resulting hydrogel is then flash frozen using liquid nitrogen and lyophilized overnight yielding aerogel particles that do not mix or get wetted upon prolonged contact with water or gastric fluid (pH<2).

The formation of a hydrophobic aerogel allows for the incorporation of discernable amounts of water absent locations within patches. These latter become hydrated upon contact with digestive fluids such as simulated intestinal fluid and bile, forming a change that is observable under ultrasound as evidenced by FIG. 9a.

5a.2 Incorporation of the Alginate Cholate Aerogel Particles into Hydrogels

### PNHEA Hydrogel as a Third Non-Degradable Hydrogel:

Four of the above 50 μL resulting materials are then placed on a rectangular Teflon mold of 7×1.5×0.2 cm which is subsequently filled with 1500 μL of a solution comprising a polymerizable mix composed of 2 mL NHEA, 2 mL MilliQ water, 53.32 μL mBAA, 301.3 μL 4.825 mg/mL 12959. Upon polymerization the resulting hydrogel layer is coated with a non-degradable backing of 1500 μL as described in example 2. The resulting patch comprises aerogel particles as sensing component within a non-degradable PHNEA hydrogel layer and a backing layer.

### PAMPS Hydrogel as a Third Non-Degradable Hydrogel:

Four of the above 50 μL resulting materials are then placed on a rectangular Teflon mold of 7×1.5×0.2 cm which is subsequently filled with 1500 μL of a solution comprising consisting of 4 mL of 50 wt % of AMPS (aliquoted from the as received stock), 20 μL of mBAA stock and 30.6 μL of Irgacure stock solution 4.825 mg/mL. Upon polymerization the resulting hydrogel layer is coated with a backing of 20 wt % Hydromed (ether based hydrophilic polyurethane preferably D4) in a solution of 95% ethanol and 5% water. The resulting patch comprises aerogel particles as sensing components within a non-degradable PAMPS hydrogel layer and a backing layer. FIG. 19(I) shows the results obtained with the present patch.

5b. Incorporation of Super Hydrophobic Lumira® Aerogel Particles as Sensing Component into Non-Degradable PAMPS Hydrogels

Super hydrophobic silica aerogel particles (Lumira®-Aerogel technologies LLC) can act as a contrast changing substance under conditions of biliary leak (FIG. 9b). More specifically 100 mg of Lumira® superhydrophobic silica aerogel particles are placed within a rectangular mold of 7×1.5×0.2 cm dimensions. This is followed by the addition of 1500 μL of a solution comprising consisting of 4 mL of 50 wt % of AMPS (aliquoted from the as received stock), 20 μL of mBAA stock and 30.6 μL of Irgacure stock solution 4.825 mg/mL. Upon a 5 minute polymerization step the PAMPS gel with incorporated Lumira aerogel particles is formed. This construct is then fused together using a 20 wt % Hydromed (ether based hydrophilic polyurethane preferably D4) in a solution of 95% ethanol and 5% water to provide a sensing patch containing Lumira® aerogel particles as sensing component embedded into a PAMPS hydrogel layer and a backing layer. The resulting semi-encapsulated sensing patch, before further use, is left to rest at 40° C. and within a fitting chamber allowing excess/residual ethanol to be evaporated. FIG. 19(II) shows the results obtained with the present patch).

### 6. Preparation of a Patch According to the Present Invention Comprising a Backing Layer and Gas Vesicle Sensing Components

6a. Preparation of a Patch Containing PAMPS Hydrogel (Hydrogel with High Relative Swelling Rate) as a First Gel, PAAm (Hydrogel with Low Relative Swelling Rate) Hydrogel as a Second Hydrogel, Gas Vesicle Sensing Components Embedded the Second Hydrogel, and PNHEA Backing Layer.

Patches were formed via layering and sequential formation of each component. Each component was then added and cured into Teflon molds.

A milky suspension of gas-containing vesicles (Halo) in PBS buffer with a typical optical density (OD) of 18 was used to dilute acrylamide monomers at 20% vol. A hydrogel precursor composition comprising 20% vol gas-containing vesicle as sensing components was made by diluting 1 mL of PBS gas-containing vesicle suspension with 4 mL of MilliQ water. To that is added 1 g of acrylamide, 64.8 μL of mBAA 2 wt % solution and 500 μL polymerization initiator Irgacure D-2959. From the resulting mix, 5 μL were placed in a periodic arrangement of interest on Teflon molds (2 cm diameter, 0.2 cm thickness). The drops were then polymerized for 5 min using a UVASPOT 400/T mercury lamp at a distance of 30 cm from the source. The light source was also equipped with a filter (H2) allowing the spectrum interval from 300 nm till the visible to reach the polymerizable mix.

With the first discrete element layer prepared, a monomer mix consisting of 4 mL of 50 wt % of AMPS (aliquoted from the as received stock), 20 μL of mBAA stock and 30.6 μL of Irgacure stock solution, was prepared and used to add 300 UL of this latter on the polymerized PAAm hydrogel containing the sensing components and the Teflon mold. After allowing the solution to settle for 1 min, the novel layer was polymerized as previously described for 5 min.

With the hydrogel layer containing sensing components, in place, the same procedure was followed for the formation of the backing. Thus, using a 300 μL backing mix coming from a polymerizable stock solution composed of pure, inhibitor removed, 2 mL NHEA monomer, mixed with 2 mL of milliQ water, 53.32 μL of mBAA 2 wt % stock solution and 302 μL of the previously prepared Irgacure solution, the monomer mix was spread on the underlying PAMPS support layer and left to diffuse in the support layers for 1 min before a 5 min polymerization step.

The gas vesicles within the patches allow for the obtention of a discrete ultrasound signal that turn's off when the patch comes in contact with simulated intestinal fluid as shown by FIG. 7.

6b. Preparation of a Patch Containing PAMPS Hydrogel (Hydrogel with High Relative Swelling Rate) as a First Gel, PAAm-Hydrogel (Hydrogel with Low Relative Swelling Rate) as a Second Hydrogel, Gas Vesicle Sensing Components Embedded the Second Hydrogel, and PAAm Backing

In a similar fashion to example 6a, the backing layer is replaced by 300 μL of polyacrylamide polymerizable backing mix as defined in example 2a and 2b.

6c. Preparation of a Patch Containing PAMPS Hydrogel (Hydrogel with High Relative Swelling Rate) as First Gel, PAAm Hydrogel (Hydrogel with Low Relative Swelling Rate) as Second Gel, and Gas-Containing Vesicles Embedded within the Second Gel, and Hydromed D4 as Backing Layer

Patches were formed via layering and sequential formation of each component. Each component was then added and cured into Teflon molds.

Sensing elements were prepared first in which gas-containing vesicles (Halo) were presented as milky suspensions in PBS buffer with a typical optical density (OD) of 18. This suspension was then used to dilute acrylamide monomers at 20% vol. A precursor composition for a hydrogel comprising 20% vol gas-containing vesicle as sensing components was made by diluting 1 mL of PBS gas-containing vesicle suspension with 4 mL of MilliQ water. To that is added 1 g of acrylamide, 64.8 μL of mBAA 2 wt % solution and 500 μL polymerization initiator Irgacure D-2959. From the resulting mix, 5 μL drops were placed in a periodic arrangement of interest on the hydromed coated Teflon molds (2 cm diameter, 0.2 cm thickness). The drops were then polymerized for 5 min using a UVASPOT 400/T mercury lamp at a distance of 30 cm from the source. The light source was also equipped with a filter (H2) allowing the spectrum interval from 300 nm till the visible to reach the polymerizable mix.

With the first discrete element layer prepared, a monomer mix consisting of 4 mL of 50 wt % of AMPS (aliquoted from the as received stock), 20 μL of mBAA stock and 30.6 μL of Irgacure stock solution, was prepared and used to add 300 μL of this latter on the polymerized sensing elements and the Teflon mold. After allowing the solution to settle for 1 min, the novel layer was polymerized as previously described for 5 min.

Finally, 300 μL of a 20 wt % Hydromed (ether based hydrophilic polyurethane preferably D4) in a solution of 95% ethanol and 5% water were used to coat the first gel layer containing the gas vesicle sensing elements. The resulting patch was then ready for further use.

7. Preparation of a First Gel Comprising ZnO Nanoparticles as Sensing/Therapeutic Component Embedded within Polyacrylamide (PAAm) for the First Portions of the Patch Claimed Herein

Flame spray pyrolysis synthesized ZnO nanoparticles were suspended in a 20 wt % acrylamide solution at a concentration of 2.5 mg/mL. For a 5 mL stock solution 54 μL of mBAA 2% solution and 8 μL TEMED polymerization accelerator were added and mixed. The resulting (prepolymer) solution was placed under sonication in order to homogenously disperse the particles in the (prepolymer) mixture. 150 μL of this mixture were placed in a Teflon mold. Then, polymerization was started by adding 5.8 μL of a 60 mg/mL ammonium persulfate MilliQ water solution and the mixture was placed at 60° C. for 4 min. The resulting opaque white hydrogel was lifted with Teflon tweezers, covered with parafilm to avoid drying and stored at 2-8° C. for further use. The results obtained with the present gel are shown by FIG. 6.

### 8. Preparation of a First Gel Comprising Commercial Ultrasound Contrast Agent Microbubbles as Sensing Component.

8a. Preparation of a First Gel Containing SonoVue® Microbubbles Embedded within Polyacrylamide (PAAm)

A SonoVue® microbubbles containing hydrogel comprising of 8 μL/mL SonoVue® was prepared as follows: 5 mL of 20 wt. % acrylamide solution were combined with 54 μL mBAA 2% solution and 375 μL of photoinitiator LAP (6.55 mg/mL). The purchased SonoVue® microbubbles were rehydrated using the prepared acrylamide solution. The SonoVue® containing hydrogel mixture was filled into custom molds of 180 μL volume and formed under a UV lamp (2×6W-365 nm VL-206.BL lamp). The encapsulated SonoVue® microbubbles can be imaged under ultrasound. Either by traditional backscatter imaging (emitted frequency equalling received frequency) or by second harmonic imaging (emitted frequency equalling half the received frequency). In both cases dissolution of SonoVue® upon contact with certain biological fluids results in a “turn-off” type signal as shown by FIG. 8a.

8b. Preparation of a First Gel Containing SonoVue® Microbubbles Embedded within Polyacrylamide (PAAm)

The purchased SonoVue® microbubbles were prepared according to the packaging instructions. This yields 5 mL of rehydrated SonoVue® in a NaCl solution. 0.25 g acrylamide was directly dissolved into 1 mL of the SonoVue® suspension. Additionally 10.8 μL of mBAA 2% solution and 75 μL of photoinitiator LAP (6.55 mg/mL) were added to the solution. 100 μL of the prepared mixture was filled into cylindrical Teflon molds and formed under a UV lamp (2×6W-365 nm VL-206.BL lamp) resulting in “pure” SonoVue® Acrylamide hydrogels.

An additional acrylamide hydrogel precursor composition was prepared containing 5 mL of 20 wt. % acrylamide together with 54 μL mBAA 2% solution and 375 μL of photoinitiator LAP (6.55 mg/mL). Into cylindrical Teflon molds 80 μL of the acrylamide hydrogel precursor composition was added. In addition either 180 μL, 80 μL or 20 μL of the SonoVue® suspension prepared according to packaging instructions was added to every mold. The gels were formed under a UV lamp (2×6W-365 nm VL206.BL lamp) resulting in the “diluted” SonoVue® Acrylamide hydrogels. Each gel was stored in an Eppendorf tube at room temperature. After 12 days the remaining ultrasonic response of SonoVue® was verified with a Clarius C3HD ultrasound probe controlled by an iPad. An Acrylamide hydrogel phantom served as a sample holder during ultrasound imaging. MilliQ water was used as an acoustic coupling medium. The results are shown by FIG. 8b.

9. Preparation of a First Gel Comprising Tantalum Oxide (Ta₂O₅) Nanoparticles as Sensing Component.
9a. 5 wt % Ta₂O₅ Nanoparticles Embedded within a PAMPS Hydrogel

A polymerizable mix composed of PAMPS gel mixture of 4 mL of 50 wt. % AMPS in water was combined with 20 μL mBAA 2% solution and 30.6 μL of photoinitiator Irgacure 2959 (4.825 mg/mL). The resulting mix was combined with Ta₂O₅ nanoparticle powder at 5 wt %. The suspension was vigorously vortexed, placed on a Teflon mold at increments of 10-150 UL and polymerized under a UV lamp (2×6W-365 nm VL-206.BL lamp). The results obtained with said gel are shown by FIG. 15a.

9b. 5 wt % Ta₂O₅ Nanoparticles Embedded within a PNAGA Hydrogel

A N-acryloyl glycinamide polymerizable mix composed of: 2 g N-acryloyl glycinamide, dissolved in 4 mL milliQ water alongside 450 μL 6.33 mg/mL Lithium phenyl-2,4,6-trimethylbenzoylphosphinate was prepared. The resulting mix was combined with Ta₂O₅ nanoparticle powder at 5 wt %. The suspension was vigorously vortexed, placed on a Teflon mold at increments of 10-150 UL and polymerized under a UV lamp (2×6W-365 nm VL-206.BL lamp). The results obtained with said gel are shown by FIG. 15b.

### 10. Preparation of a Patch According to the Present Invention Containing a First Gel and a Second Gel Having Different Relative Swelling Ratios for the Detection of Gastric Fluid Under Computer Tomography X-Rays.

10a. Preparation of a First Gel Janus Cylindrical Sensing Element Containing Ta₂O₅ Nanoparticles Embedded within a Non-Degradable PAMPS Hydrogel and Ta₂O₅ Nanoparticles Embedded Non-Degradable PNAGA Hydrogel Placed within a Nondegradable Polyacrylamide Hydrogel

To a cylindrical polystyrene mold (diameter 0.6 cm and height 1.5 cm) are added 150 μL of polymerizable stock solution from a 6 mL stock solution containing 33 wt % NAGA (N-acryloyl glycinamide) in MilliQ water, 450 μL of 6.33 mg/mL initiator solution (LAP) and a 5 wt % overall tantalum oxide nanoparticle powder. The mix was polymerized for 5 min under a UV lamp (2×6W-365 nm VL-206.BL lamp).

To the formed cylindrical 150 μL PNAGA 33 wt %, 5 wt % Ta₂O₅ hydrogel are added directly in the same mold 150 μL of a polymerizable stock composed of 4 mL 50 wt % AMPS, 20 μL mBAA 2 wt % and 30.6 μL of 4.825 mg/mL Irgacure 2959, tantalum oxide at a quantity of 5 wt % overall. The resulting patches then comprise a PAMPS hydrogel having a high relative swelling ratio containing CT active contrast agent tantalum oxide, within a PNAGA hydrogel having a low relative swelling ration equally containing tantalum oxide. These two layers act differently in the presence of digestive fluid yielding a discernable signal before and after contact with bodily fluids (FIG. 16a) and can be used as a patch according to a second aspect of the present invention.

The resulting cylindrical gel is then placed within a rectangular teflon mold of dimensions 5×3×1.5 cm and this latter is filled with 2 mL of a polymerizable 20 wt % acrylamide mix consisting of: 5 mL of 20 wt % Acrylamide, 500 μL 4.825 mg/mL Irgacure D-2959, 64.8 μL mBAA 2 wt % (all solutions were in MilliQ water) to provide a patch according to the second embodiment of the present invention.

10b. Rectangular Patch Containing Non-Degradable PAMPS Hydrogel as a First Gel, Non-Degradable PNAGA Hydrogel as a Second Hydrogel and Tantalum Oxide Nanoparticles Embedded within the First Gel and the Second Gel

To a stock solution composed of 4 mL 50 wt % AMPS, 20 μL mBAA 2 wt % and 30.6 μL of 4.825 mg/mL Irgacure 2959, tantalum oxide nanoparticles powder (Sigma, size 10-500 nm) are added at a quantity of 5 wt % overall. The mix is vigorously shaken and then is casted into rectangular molds (3×1.5×0.2 cm) molds at 50 μL increments (3×) and polymerized for 5 min using a UVASPOT 400/T mercury lamp at a distance of 30 cm from the source, equipped with an H2 filter. The resulting pattern of shaped disk or hemispherical PAMPS hydrogels are embedded within 1000 μL liquid polymerizable layer of a 6 mL stock solution containing 33 wt % NAGA (Nacryloyl glycinamide) in MilliQ water, 450 μL of 6.33 mg/mL initiator solution (LAP) and a 5 wt % overall tantalum oxide nanoparticle powder. The resulting system was polymerized for 5 min under a UV lamp (2×6W-365 nm VL-206.BL lamp) The resulting patches then comprise a PAMPS hydrogel portion (hydrogel with high relative swelling ratio) containing CT active contrast agent tantalum oxide, within a PNAGA hydrogel (hydrogel with low relative swelling ratio) equally containing tantalum oxide. These two hydrogels act differently in the presence of digestive fluid yielding a discernible signal before and after contact with bodily fluids (FIG. 16b).

### 11. Preparation of a Patch According to the Present Invention Comprising Combination of High and Low Swelling Ration Matrix for the Detection of Gastric Fluid Under Both Computer Tomography X-Rays and Ultrasound.

11a. Dog Bone Patch Containing a PAAm Hydrogel as a First Gel Having Embedded Therein Gas Vesicles as Sensing Components, and a PNAGA Hydrogel as a Second Hydrogel Having Embedded Therein Tantalum Oxide Nanoparticle as Sensing Components

To a dog bone shaped Teflon mold of dimensions 3 cm in length and 0.5 cm in junction width are added 50 μL of 40 vol % Halo gas vesicles polymerizable solution diluted by an appropriate volume originating from a 5 mL stock of 20 wt % Acrylamide, 500 μL 4.825 mg/mL Irgacure D-2959, 64.8 μL mBAA 2 wt % (all solutions were in MilliQ water). The 50 μL Halo GV hydrogel is formed after 5 min irradiation under a under a UVASPOT 400/T mercury lamp at a distance of 30 cm from the source equipped with an H2 filter.

The mold is then filled with a partially overlapping quantity of 50 μL a 6 mL stock solution containing 33 wt % NAGA (N-acryloyl glycinamide) in MilliQ water, 450 μL of 6.33 mg/mL initiator solution (LAP) and a 5 wt % overall tantalum oxide nanoparticle powder and polymerized for 5 min under a UV lamp (2×6W-365 nm VL-206.BL lamp). The resulting dog bone patch allows simultaneous detection under ultrasound and CT modalities (FIG. 18a) and the obtention of a disappearing signal under ultrasound in the case of contact with digestive biological fluids such as simulated intestinal fluid.

11b. Rectangular Patch Containing a PAAm Gel as a First Gel Having Embedded Therein Gas Vesicles as a Sensing Component and PNAGA Gel as a Second Gel Having Embedded Therein Ta₂O₅ Nanoparticles as a Sensing Component

To a stock solution composed of 40 vol % Halo gas-containing vesicles polymerizable solution diluted by an appropriate volume originating from a 5 mL stock of 20 wt % Acrylamide, 500 μL 4.825 mg/mL Irgacure D-2959, 64.8 μL mBAA 2 wt % (all solutions were in MilliQ water) into rectangular molds (3×1.5×0.2 cm) molds at 50 μL increments (3×) and polymerized for 5 min using a UVASPOT 400/T mercury lamp at a distance of 30 cm from the source, equipped with an H2 filter. The resulting pattern of shaped disk or hemispherical hydrogels are coated with 1000 μL of a 6 mL stock solution containing 33 wt % NAGA (N-acryloyl glycinamide) in MilliQ water, 450 μL of 6.33 mg/mL initiator solution (LAP) and a 5 wt % overall tantalum oxide nanoparticle powder. The resulting suspension was homogenized before addition of the mix to the rectangular patterned mold and polymerized for 5 min under a UV lamp (2×6W-365 nm VL-206.BL lamp) The resulting patch allows simultaneous detection under ultrasound and CT modalities (FIG. 18b) and the obtention of a disappearing signal under ultrasound in the case of contact with digestive biological fluids such as simulated intestinal fluid.

12. Preparation of Patches with Discernible Signal Variations Under MRI Upon Contact with Digestive Fluids.
12.a Iron Oxide Nanoparticles within a Digestible PNHEA-BAC Hydrogel Housed by PNHEA Non-Digestible Hydrogel.

To a premade sheet of dimensions of 1.5×1×0.2 cm of medical polyurethane hydromed D4 made from a mix consisting of 20 wt % Hydromed (ether based hydrophilic polyurethane preferably D4) in a solution of 95% ethanol and 5% water are added premade NHEA-BAC hydrogels composed of 10 wt % iron oxide nanoparticles (50-200 nm in size and nanoflower shaped) bare or functionalized. The hydrogel matrix housing the nanoparticles is made of an indicative stock of 0.25 mL of purified NHEA, 77.5 μL of 2 wt % BAC (NN-Bis(acrylyol) cystamine) crosslinker, 1.6 μL of TEMED accelerator, to which for a gel 50 μL are added 1.8 μL of a 60 mg/mL solution of APS. The resulting patterned flexible sheet of polyurethane is then fused together into a single material using a polymerizable mix composed of 2 mL NHEA, 2 mL MilliQ water, 53.32 μL mBAA, 301.3 μL 4.825 mg/mL 12959. The resulting patches are then ready for interaction with biological fluids or can be further processed.

12b. Patch Containing a PAMPS Hydrogel (Hydrogel with High Swelling Ratio) as a First Non-Degradable Hydrogel Having Embedded Therein Iron Oxide Nanoparticles as Sensing Component and a Non-Degradable PNAGA Hydrogel (Hydrogel with Low Swelling Ratio) as a Second Non-Degradable Hydrogel Having Embedded Therein Tantalum Oxide Nanoparticles as Sensing Component

To a stock solution composed of 4 mL 50 wt % AMPS, 20 μL mBAA 2 wt %, iron oxide nanoparticles powder (8-200 nm) are added at a quantity of 10 wt % overall. The mix is vigorously shaken and sonicated and then is casted into round circular molds of 0.2 to 0.5 cm diameter and 0.2 cm thickness (amounts corresponding to 50-150 μL) for which and for an amount of 50 μL an amount of 5.6 μL of 60 mg/mL APS are added to form gels. The resulting shaped disk or hemispherical hydrogels are placed and arranged in a desired pattern onto a larger rectangular Teflon mold 1.5×1×0.2 cm. To this patterned mold are added 300-1000 μL of a 6 mL stock solution containing 33 wt % NAGA (N-acryloyl glycinamide) in MilliQ water, 450 μL of 6.33 mg/mL initiator solution (LAP) (or 4.825 mg/mL Irgacure 2959 or an equivalent amount of ammonium persulfate combined with accelerator TEMED) and a 5 wt % overall tantalum oxide nanoparticle powder. The resulting patches are then ready for interaction with biological fluids or can be further processed.

12c. Patch Containing a Non-Degradable PAMPS Hydrogel Having Embedded Therein Iron Oxide Nanoparticles as Sensing Component, a Non-Degradable PAAm Hydrogel Having Embedded Therein Gas Vesicles as Sensing Component, and a Polyurethane Hydrogel

To a stock solution composed of 4 mL 50 wt % AMPS, 20 μL mBAA 2 wt %, iron oxide nanoparticles powder (8-200 nm) are added at a quantity of 10 wt % overall. The mix is vigorously shaken and sonicated and then is casted into round circular molds of 0.2 to 0.5 cm diameter and 0.2 cm thickness (amounts corresponding to 50-150 μL) for which and for an amount of 50 μL an amount of 5.6 μL of 60 mg/mL APS are added to form gels. The resulting shaped disk or hemispherical hydrogels are placed and arranged in a desired pattern onto a larger rectangular Teflon mold 1.5×1×0.2 cm which contains a 0.1 cm layer of medical polyurethane hydromed D4 made from a mix consisting of 20 wt % Hydromed (ether based hydrophilic polyurethane preferably D4) in a solution of 95% ethanol and 5% water. To this patterned sheet are added 300-1000 μL of a stock solution of Ana or Halo gas vesicles 40 vol % within a polymerizable mix of made of 5 mL of 20 wt % Acrylamide, 500 μL 4.825 mg/mL 12959 (or 500 μL 6.33 mg/mL LAP) 64.8 μL mBAA 2 wt %. The resulting patches are then ready for interaction with biological fluids or can be further processed.

12d. Patch Containing a Non-Degradable PHNEA Hydrogel, Non-Degradable PAAm Hydrogel Having Embedded Therein a Gadolinium Chelate as Sensing Component, and a Non-Degradable Polyurethane Hydrogel

To a stock solution composed of 5 mL of 20 wt % Acrylamide, 64.8 μL mBAA 2 wt % 2 wt %, made in water containing 1 mmol/mL are added iron oxide nanoparticles in powder form (bare or functionalized with gadolinium-DTPA via cleavable linker, particle size 8-200 nm) are at a quantity of 10 wt % overall. The mix is vigorously shaken and sonicated and then is casted into round circular molds of 0.2 to 0.5 cm diameter and 0.2 cm thickness (amounts corresponding to 50-150 μL) for which and for an amount of 50 μL an amount of 5.6 μL of 60 mg/mL APS are added to form gels. The resulting shaped disk or hemispherical hydrogels are placed and arranged in a desired pattern onto a larger rectangular Teflon mold 1.5×1×0.2 cm which contains a 0.1 cm layer of medical polyurethane hydromed D4 made from a mix consisting of 20 wt % Hydromed (ether based hydrophilic polyurethane preferably D4) in a solution of 95% ethanol and 5% water. To this patterned sheet are added 300-1000 μL of a stock solution composed of 2 mL NHEA, 2 mL MilliQ water, 53.32 μL mBAA, 301.3 μL 4.825 mg/mL 12959 are added and upon polymerization fuse the patch together. The resulting patches are then ready for interaction with biological fluids or can be further processed.

### Swelling Ratio Determination Experiments

The relative swelling ratio Rᵣₑₗ is defined as,

$${R}_{r e l} = \frac{{M}_{s}}{{M}_{i}}$$

Where Mₛ is the mass of the hydrogel/xerogel/cryogel/aerogel sample after swelling for a given time point, defined here as 24 h. Mᵢ is the initial mass of the hydrogel sample hydrogel/xerogel/cryogel/aerogel prior to incorporation into the final assembled patch/item. Initially 50 μL formed gel formulations were transferred into vials of 15 mL. Then (5 mL) of fresh simulated intestinal fluid were added respectively to investigate the formulation's swelling. Finally, the vials containing the items along with the incubating fluids were transferred onto a shaker (Titramax 101, Heidolph, 200 rpm) and incubated at 37° C. with shaking. The mass of the hydrogels was deduced by measuring the residual mass of the vial after residual fluid removal and crossreferencing it with mass of the formulation itself for low-swelling materials. The final swelling ratio was considered to be the 24 h time point.

### Simulated Intestinal Fluid Preparation

Simulated intestinal fluid was prepared using lyophilized porcine pancreatin powder (>8 USP) and a protocol from the United States pharmacopoeia (Test Solutions, United States Pharmacopeia 30, NF 25, 2007). In brief, (6.8 g, 50 mmol) monobasic potassium phosphate was dissolved in 250 mL milliQ water. To this solution, (77 mL 0.2 mol/L) sodium hydroxide solution and 500 mL milliQ water were added and mixed along with (10 g) pancreatin (from porcine pancreas, 8 USP units activity/g). The SIF/P suspension was adjusted to pH 6.8 with either 0.2 mol/L sodium hydroxide or 0.2 mol/L hydrochloric acid and diluted with water to 1000 mL.

Swelling ratio table:

| | Average Swelling | Average Swelling |
|---|---|---|
| Matrix formulation | Ratio in SIF | Ratio in PBS |
| PAMPS 50 wt % | 42 | 39 |
| PNAGA 33 wt % | 2 | 2 |
| PNHEA 50 wt % | 11 | 11 |
| P(AA-MA) 50 wt % | 38 | 2 |
| P(Aam-MA-AA) 50 wt % | 28 | 8 |
| PAAm 20 wt % | 5 | 5 |
| Hydromed D4 | 1 | 1 |
| Lumira aerogel | 1 | 1 |
| PNHEA 25 wt % - BAC | digested | |
| BSA hydrogel | digested | |
| Alginate cholate aerogel | digested | 1 |

### Simulated Gastric Fluid (SGF)

Simulated gastric fluid was made using simplified guidelines from the United States pharmacopeia excluding pepsin. More specifically, a 35 mM NaCl solution was prepared from distilled water. The solution was then adjusted to pH=2.0 using 0.1M HCl and the resulting fluid was used straight away.

### Bile Fluid

Lyophilized Ox-bile was purchased from Sigma-Aldrich and was reconstituted using milliQ water. (1 g of bile powder was added 7 mL of MilliQ water).

1. A patch for detecting by at least one non-invasive imaging technique a leak of a biological fluid at an application site, said patch comprising:
i) a layer comprising
one or more first portions containing a first gel;
one or more second portions containing a second gel; and
at least one sensing component embedded within the first gel, the second gel, or the first gel and the second gel;
the at least one sensing component being selected from gas-containing vesicles, gas-containing microbubbles, salt containing particles, metal or metal oxide nanoparticles, carbide nanoparticles, radio-opaque iodinated or brominated organic compounds, gadolinium complexes, monosaccharide containing microspheres, polysaccharide containing microspheres, protein containing microspheres, poly(vinylpyrrolidone)-iodine complex particles, and combinations thereof;
the first gel and the second gel being selected from the group consisting of a hydrogel, an aerogel, and a xerogel, and comprising a synthetic polymer, a natural polymer, or a mixture thereof; and
wherein
i-1) the first gel is non-degradable and the second gel is degradable, or
i-2) the first gel and the second gel are non-degradable, the first gel has a relative swelling ratio, and the second gel has a relative swelling ratio, and the relative swelling ratio of the first gel is different from the relative swelling ratio of the second gel, with the relative swelling ratio being the ratio between the mass of said gel after incubation for 24 h at 37° C. in a simulated intestinal fluid and the mass of said gel prior to incubation in the simulated intestinal fluid;
or
ii) a layer containing a third gel comprising a synthetic polymer, a natural polymer, or a mixture thereof, and being a hydrogel, and
at least one sensing component embedded within said third gel,
the at least one sensing component being selected from the group consisting of aerogel particles, and xerogel particles,
wherein
ii-1) the at least one sensing component is degradable and the third gel is a non-degradable,
ii-2) the at least one sensing component is non-degradable, the third gel is nondegradable, the at least one sensing component has a relative swelling ratio and the third gel has a relative swelling ratio, and the relative swelling ratio of the sensing component is different from the relative swelling ratio of the third gel, with the relative swelling ratio being the ratio between the mass of said at least one sensing component, or of said third gel after incubation for 24 h at 37° C. in a simulated intestinal fluid and the mass of said at least one sensing component, or of said hydrogel prior to incubation in the simulated intestinal fluid, or
ii-3) the at least one sensing component is non-degradable and the third gel is degradable.

2. The patch according to claim 1, wherein the at least one noninvasive imaging technique is selected from ultrasound imaging, computer tomography, magnetic resonance imaging, magnetic particle imaging and radio-frequency identification.

3. The patch according to claim 1, wherein the at least one sensing component is embedded within the first gel and the second gel, and the at least one sensing component embedded within the first gel is different from the at least one sensing component embedded within the second gel.

4. The patch according to claim 1, wherein the first gel and/or the third gel is a non-degradable hydrogel.

5. The patch according to claim 1, wherein the at least one non-invasive imaging technique is ultrasound imaging.

6. The patch according to claim 5, wherein the at least one sensing component is selected from gas-containing vesicles, gas-containing microbubbles, salt containing particles, metal or metal oxide nanoparticles, magnetic carbide nanoparticles, aerogel particles, xerogel particles, and combinations thereof.

7. The patch according to claim 1, wherein the at least one non-invasive imaging technique is computer tomography.

8. The patch according to claim 7, wherein the at least one sensing component is selected from salt containing particles, metal or metal oxide nanoparticles, radio-opaque iodinated or brominated organic compounds, aerogel particles, xerogel particles, poly(vinylpyrrolidone)-iodine complex particles, and combinations thereof.

9. The patch according to claim 7, wherein the at least one sensing component is selected from barium sulphate particles and tantalum oxide nanoparticles.

10. The patch according to claim 1, wherein the at least one non-invasive imaging technique is magnetic particle imaging and the at least one sensing component is selected from magnetic metal or metal oxide nanoparticles, magnetic carbide nanoparticles, and combinations thereof thereof.

11. The patch according to claim 10, wherein the magnetic metal or metal oxide nanoparticles, or the magnetic carbide nanoparticles are linked to the synthetic polymer and/or natural polymer via a hydrolytically cleavable linker and/or an enzyme-digestible peptide.

12. The patch according to claim 1, wherein the at least one non-invasive imaging technique is magnetic resonance imaging.

13. The patch according to claim 12, wherein the at least one sensing component is selected from magnetic metal or metal oxide nanoparticles, optionally functionalized with a T1 contrast agent, magnetic carbide nanoparticles, optionally functionalized with a T1 contrast agent, gadolinium complexes, and combinations thereof.

14. The patch according to claim 1, wherein the patch further comprises a backing layer, optionally containing sensing components.
